(19) United States
(12) Patent Application Publication
(10) Pub. No.: US 2026/0185050 A1
(43) Pub. Date: Jul. 2, 2026

(54) BIOLOGIC INTERFACIAL TISSUE-ENGINEERED SYSTEMS USEFUL AS ARTHROPOD BITEMODEL

(71) Applicant: UNIVERSITY OF CENTRAL FLORIDA RESEARCH FOUNDATION, INC., Orlando, FL (US)

(72) Inventors: Bradley Jay WILLENBERG, Orlando, FL (US); Thomas KEAN, Orlando, FL (US); Mollie JEWETT, Orlando, FL (US); Robert STEWARD, Jr., Orlando, FL (US); Mona MATHEW, Orlando, FL (US)

(21) Appl. No.: 19/131,322

(22) PCT Filed: Dec. 11, 2023
(86) PCT No.: PCT/US2023/083347

§ 371 Date: May 20, 2025

Related U.S. Application Data
(60) Provisional application No. 63/431,361, filed on Dec. 9, 2022.

(51) Int. Cl. C12N 5/077; B33Y 40/20; B33Y 80/00; C12N 5/071

(52) U.S. Cl. CPC C12N 5/0656; B33Y 40/20; B33Y 80/00; C12N 5/069; C12N 2503/04; C12N 2513/00; C12N 2533/54; C12N 2533/74; C12N 2537/10

(57) ABSTRACT

Biologic interfacial tissue-engineered systems (BITES) disclosed here is a biomaterial platform to provide engineered human and/or animal skin-like constructs for study of biting and/or blood feeding behavior of mosquitoes as well as other insects and arthropods. Provided herein are use and methods of making for BITES, in particular use and methods of cellularized Capgel for BITES with human cells.

### FIELD OF THE INVENTION

The invention relates to a biomaterial scaffold, for example, a scaffold made of capillary alginate gel (Capgel) for biologic interfacial tissue-engineered system (BITES) for use as a new platform for study of arthropod biting behavior.

### BACKGROUND OF THE INVENTION

Arthropods such as insects, myriapods (e.g., centipedes and millipedes), chelicerates (e.g., ticks, spiders, mites and scorpions), and crustaceans (e.g., prawn and crabs), can inflict bites, stings and infect human skin. They can carry myriad of pathogens to transmit diseases, and their bites give rise to allergic conditions such as hay fever, asthma and atopic eczema. Among them, insects are the largest group within the arthropod phylum, and mosquitoes are one of the families of insects, called Culicidae including almost 3,600 species of small flies.

Mosquitoes, especially female mosquitoes bite to feed blood for reproduction, and in their blood feeding process, they ingest pathogens and transmit them to other hosts. In this way, many species of mosquitoes are important vectors of parasitic diseases such as malaria and filariasis, and arboviral diseases such as yellow fever, Chikungunya, West Nile, dengue fever, and Zika, which sometimes cause deadly outcome.

However, in spite of such risk of transmitting pathogens, many aspects of biting/blood feeding behavior of mosquitoes as well as other medically important arthropods, such as ticks, are not well understood, for example, their preferred human skin condition, selection of landing site and biting site on human skin, feeding avidity (i.e., number of feeding attempts), feeding time, the quantity of the blood they take in blood feeding process, etc.

Vector-borne diseases caused by pathogens such as parasites, bacteria, and viruses are responsible for more than 700,000 deaths annually [1]. Worldwide, major outbreaks of malaria, dengue virus (DENV), and Zika virus (ZIKV) transmitted by mosquitoes have caused overwhelming healthcare burdens. There are also more than 60 million cases of lymphatic filariasis caused by filarial parasites transmitted via mosquitoes [2-4]. These hematophagous (blood-feeding) arthropod vectors acquire the pathogens that cause these diseases during the ingestion of blood meals from infected vertebrate hosts. While probing for a blood vessel, a mosquito penetrates the stylet mouthparts of its proboscis through the vertebrate epidermis into the dermis and expectorates saliva along with the pathogen into the host skin [5,6].

For the continuation of the pathogen transmission cycle from vertebrate to mosquito, the pathogen must be abundant enough throughout the vertebrate body to be taken up again in the next mosquito bite [7]. In the orchestration of initial pathogen transmission, replication at the bite site and the early host immune responses are critical events prior to dissemination throughout the host [8,9]. Therefore, these early events are important for disease outcomes and strongly correlate with the peripheral pathogen burden and mortality [9].

In vitro tools such as artificial blood-feeding systems [10-15], cell cultures [16-19], and skin tissue explants [20,21] offer cost-effective means of maintaining closed-loop laboratory mosquito colonies, enable reductionist and/or high-throughput experimental approaches and have fewer ethical considerations and limitations compared to using live animals and human volunteers. However, none of these approaches allow the comprehensive analysis of mosquito biting/feeding and the molecular and cellular events that occur in the skin.

Artificial blood-feeding systems for maintaining mosquito colonies can be as straightforward as a warmed, blood-filled collagenous (bovine) sausage casing [11,12]. More sophisticated glass feeders, first designed by Rutledge et al. [13], that use Parafilm or animal-derived membranes (e.g., collagenous bovine casing, thinned chicken skin) stretched over a reservoir of blood warmed by a circulating water jacket are also frequently used [12,14].

Sri-in et al. developed a method using Parafilm TM-M membrane packets filled with warm blood for mosquito feeding and oral infection [15]. Using 3D printing, an acellular hydrogel-based “skin” with warmed blood circulating through vessel-like structures was recently developed for automated mosquito feeding and the study of repellents [22].

As all the above systems are acellular/lack living cells, they do not offer the potential for investigating cellular biology at the bite site.

Two-dimensional (i.e., flat) cell culture models have been informative as to the impacts of mosquito salivary proteins on human skin cells and have provided insights about arboviral infection in these cells [23]. Mosquitoes, however, cannot bite and blood-feed on 2D cell cultures and thus the important interplay between the vector mouthparts, expectorated saliva, transmitted pathogens (if any), and host cells (resident and migratory) at the skin bite site cannot be appreciated. Human ex vivo skin explants preserve the cellular and structural anatomy of the skin and have been used to examine dengue and Zika virus infections [20,21]. However, the supply and maintenance of these explants in culture are limited and mosquito bite/blood-feeding on the tissue has not been examined.

Some of these shortcomings have been addressed through engineered full-thickness skin equivalents (FTSE) composed of collagen type 1 scaffolds with human keratinocytes and fibroblasts, but this model is avascular and does not include blood [24].

In addition, due to the lack of proper assay systems to measure and analyze mosquito behavior, studies often use human subjects in order to study how mosquitos feed blood. Scientists often sacrifice their own skin, allowing mosquitoes to bite them in laboratory settings to observe their feeding behavior and to quantify the number of landings and/or bites, or the time it takes to complete a blood-feeding, and manually record the score of experimental outcomes. Such use of human subjects as bait is not only unpleasant for the volunteers, but also has limitations, e.g., the number and type of experiments (e.g., no use of infected mosquitoes) as well as measurement methods, and thus innovative experimental approach models have been in need.

### SUMMARY

Together, all of the aforementioned gaps illustrate the need for in vitro-engineered 3D tissue platforms that mimic the skin/dermis with human cells and micro blood vessel structures into and from which arthropods—such as mosquitoes—can bite, probe, and blood-feed. Such tools would be powerful enablers of mosquito bite-site cellular and molecular biology investigations.

Uniquely, to fill this need, a versatile family of 3D self-assembled capillary alginate gel (Capgel) biomaterial scaffolds was previously developed and studied by our group in a range of tissue engineering settings [25-31]. Then, in this proof-of-concept study, a Biologic Interfacial Tissue-Engineered System (BITES) is developed in this disclosure, which uses human cell-lined Capgel with blood loaded into the cellularized capillary lumens.

This new in vitro platform was designed specifically to facilitate biological investigations of mosquito skin bite sites, and it is demonstrated in this disclosure that female Aedes (Ae.) aegypti mosquitoes can bite, probe, and blood-feed naturally from BITES.

The new BITES model described herein enables a study of blood-feeding behavior of infectious mosquitoes as well as other medically relevant arthropods, such as ticks, due to its multiple micro-capillary structure which allows to grow human cells and contain human body fluid inside. In addition, BITES model can be applied to testing of the effectiveness of various substances for their use as repellent or insecticide.

In a specific embodiment, the BITES model is composed of Capgel, which is a scaffold of densely-packed parallel microtubular capillary structures. In a more specific embodiment, Capgel is composed of an elastomeric hydrogel made of biopolymers such as alginate and gelatin that includes a stable matrix of peptide-bond crosslinked network of alginate polysaccharide chains and gelatin polypeptide chains.

Various Capgels have been used in wide range of tissue engineering applications including 3D stem cell culture scaffolds [Willenberg, B. J. Z. et al., J Biomed Mater Res A, 2006, 79 (2): 440-50], injectable stem cell delivery [Willenberg, B. J. Z. et al, J. Biomater. Sci. Polym. Ed., 2011, 22 (12): 1621-1637], in vitro construction of functional nerve [Anderson, W. A. et al., J Neurosci Methods, 2018, 305:46-53; George, D. S. et al., J Tissue Eng Regen Med, 2019, 13 (3): 385-395], and as injectable wound healing biomaterials [Bosak, A. et al., International Journal of Polymeric Materials and Polymeric Biomaterials, 2018. 68 (18): 1108-1117; Rocca, D. G. e al., Int J Cardiol, 2016, 220:149-54].

Capgels are a unique family of self-assembled hydrogel biomaterials characterized in that the gel comprises a plurality of parallel microtubular capillaries. The diameters and lengths of each of the microtubular capillaries may be the same, similar or vary, and the diameter of a microtubular capillary may be the same, similar or vary between one end and the other end of the capillary. Also, the cross-section of each of the microtubular capillaries is circular or non-circular. Some of the microtubular capillaries are open at both ends and the others are open at one end. The diameter of each of the parallel microtubular capillaries is within the range of about 10 um to about 300 um, in more particular between ˜50 and 70 μm.

In addition, the inside of these microstructure of parallel capillaries can be cellularized with cells, wherein the cell type may include dermal fibroblasts, vascular endothelial cells, keratinocytes, immune cells, and mesenchymal stem cells, wherein immune cells optionally comprise macrophages, either tissue-resident or derived from infiltration of monocytes, or dendritic cells. The capillaries having open ends at both sides can be connected to a peristaltic pump or height-adjusted syringes to make the cell culture medium, serum and/or blood flow through the microtubular capillaries, mimicking real physiological conditions. In addition to or independent to capillaries that have been cellularized, the capillaries may contain fluid such as cell culture medium, serum, blood,

Attractive toxic sugar baits (ATSB) are a formulation vehicle for presenting ingestible active ingredients to mosquitoes. A principle and recurring area of ATSB research are toxicants that function well as an ingestible toxin, preferably with low to no environmental ramifications. The BITES model can be used in studies to elucidate the mechanism(s) of kill for propylene glycol, an alcohol that has recently been discovered to outperform erythritol in selective dose response work in the males and females of both Aedes aegypti and Aedes albopictus and in select experiments with Culex pipiens. Accordingly, independently, or in addition to the cellularization process, the capillaries may contain fluid such as cell culture medium, serum, blood, alcohol (e.g. propylene glycol), sugar or sugar solutions, sugar alcohols (e.g. erthritol) and/or insecticides. Sugars used for loading the capillaries include, but are not limited to, sucrose, glucose, fructose, and the like.

The primary component of these hydrogels is alginate, a popular natural anionic linear polysaccharide biopolymer composed of β-D-mannuronic and α-L-guluronic acids residues. Initial ionic crosslinking of an alginate solution via uniaxial diffusion of divalent metal ions such as Cu²⁺ generates the Capgel self-assembled micro-capillary structure. Capillary diameter and density can be tailored for a given application via selection of the initial alginate and/or diffusing divalent metal ion (i.e., Cu²⁺) concentrations [Axpe, E. and M. L. Oyen, Int J Mol Sci, 2016, 17 (12): 1976; Lee, K. Y. and D. J. Mooney, Prog Polym Sci, 2012, 37 (1): 106-126].

Since alginate does not have cell attachment sites, gelatin, which has intrinsic Arg-Gly-Asp (RGD) cell-adhesion motifs, is added to the initial alginate solution [Gungor-Ozkerim, P. S. et al, Biomater Sci, 2018, 6 (5): 915-946; Neufurth, M. et al., Biomaterials, 2014, 35 (31): 8810-8819; Zhang, T., K. C. Yan, L. Biofabrication, 2013, 5 (4): 045010]. After ionic crosslinking, these gels are sectioned into blocks, subjected to carbodiimide chemistry to form peptide crosslinks to produce Capgel scaffolds.

As disclosed herein, it was demonstrated that the produced micro-capillary structure of Capgel for BITES enables robust cell seeding, growth, and colonization, including micro-vascularization under laboratory cell culture condition, and that the Capgel for BITES having human cells, such as human dermal fibroblast (HDF) and/or human umbilical vein endothelial cells (HUVEC), among others, as well as human body fluid such as human blood inside the micro-capillary structure attracts mosquitoes and induces them to land and engage in blood feeding, thereby showing that this scaffold can imitate human skin condition.

Therefore, the disclosed BITES models comprise a combination of Capgel and cells and serves as an improved platform to study biting and/or blood feeding behavior of mosquitos as well as that of other human skin-biting insects and arthropods. The disclosed BITES models also are useful for testing the effects of certain substances such as insecticides or repellants on insects and arthropods, such as by coating them on the Capgel surface using brush or spray.

For Capgel, other biopolymers can also be contemplated, such as alginate, gelatin. collagen, hyaluronic acid, chitosan, laminin, fibronectin, glycosaminoglycans, chondroitin sulfate, dermatan sulfate, proteoglycans or other extracellular matrix molecules, silicones, methacrylate hydrogels, degradable polyester biomaterials (e.g., PLA, PGA, PLLA, etc.), the like, and combination thereof.

A method of making the Capgel and a method of using the Capgel as a arthropod bite model are also disclosed here.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. Schematic overview of the BITES platform. (A) Illustration of a sterile Capgel block depicting the patent capillary microstructure. (B) Loading/charging of the Capgel with either human dermal fibroblasts (HDFs) or human umbilical vein endothelial cells (HUVECs) into the capillaries of the block. (C) Culture of the human cell-laden Capgel to cellularize the block. (D) Loading ofblood into the capillaries of the cellularized Capgel to create a BITES construct. (E) Presentation of the BITES construct to female Aedes (Ae.) aegypti mosquitoes (prototypic arthropod) for natural biting, probing, and blood-feeding. Inset: Zoomed-in depiction of a mosquito with the stylet inserted into the BITES construct, engaging in natural biting/probing/blood-feeding. (F) Transfer of the BITES construct from the mosquito cage into an incubator for (optional) post biting/probing/blood-feeding cell culture. (G) Retrieval of the BITES construct following post-biting/probing/blood-feeding cell culture (if any) for processing and raftview.

FIG. 2. Female Ae. aegypti mosquitoes bite into, probe and blood-feed from Capgel loaded with blood in a manner matching the natural taking of a blood meal from a vertebrate host. (A) Representative differential interference contrast (DIC) micrograph of a blood-loaded Capgel showing the end-on view of the capillary microstructure, termed capview; RBCs within the capillaries and those that have flowed out are observable as multiple small, dark, circular/spherical particles throughout the image. (B) Representative DIC micrograph of a blood-loaded Capgel (similar to that of (A)) with the long-axis of the capillary in the plane of the image, which is termed raftview; RBCs are now observable as multiple small, dark, circular/spherical particles confined exclusively within capillaries. (C—H) Nonconsecutive, sequential set of image stills taken from video recordings capturing the same representative taking of a blood meal event by an Ae. aegypti female from a warmed, blood-loaded Capgel block oriented in raftview: (C) biting and probing; (D) initiation of blood ingestion; (E-H) feeding to repletion, including prediuretic droplet excretion. (I) Plot of abdominal width ratio (Equation (1)) and the number of excreted prediuretic droplets over time of the blood meal taken by a female Ae. aegypti mosquito from warmed, blood-loaded Capgel. White asterisks label the warmed, blood-loaded Capgel; black asterisks label the polystyrene foam insulation surrounding the Capgel block. White lines mark the abdominal width of the mosquito and greencircles enclose prediuretic droplet(s). The green number is the estimated total volume (including prediuretic excretions) of blood ingested, calculated by approximating the abdomen initial and final volumes as ellipsoids (Equation (2)), taking the difference (Equation (3)), and adding the droplet volumes approximated as spheres (Equation (4)); the red number is the estimated final volume of blood in the midgut only. Scale bars=50 μm for (A,B) and 1 mm for (C—H).

FIG. 3. HDFs and HUVECs line scaffold capillaries with tissue when cultured within Capgel biomaterials for four (4) weeks. (A,B) Representative DIC micrographs of Capgel imaged in capview and raftview orientations, respectively. (C,D) Representative maximum z-projection confocal fluorescence micrographs of HDF tissue structures formed within Capgel scaffold cultures imaged in capview and raftview orientations, respectively. (E,F) Representative maximum z-projection confocal fluorescence micrographs of HUVEC tissue structures formed within Capgel scaffold cultures imaged in capview and raftview orientations, respectively. Green fluorescence: Actin Green 488 nm cytoskeletal staining; blue fluorescence: Nucblue nuclei staining. Scale bars=100 μm for (A,B) and 50 μm for (C-F).

FIG. 4. HDFs and HUVECs form stable, tubular 3D tissue structures morphologically similar to microvessels when cultured for four (4) weeks within Capgel biomaterial scaffolds. (A,B) Representative raftview maximum z-projection confocal fluorescence micrograph and corresponding reconstructed orthogonal capview image, respectively, of tubular HDF tissue structures formed within Capgel scaffolds. (C,D) Representative raft- and capview snapshots, respectively, from confocal fluorescence microscopy 3D renderings of tubular HDF tissue formations within Capgel. (E,F) Representative raftview maximum z-projection confocal fluorescence micrograph and corresponding reconstructed orthogonal capview image, respectively, of tubular HUVEC tissue structures formed within Capgel scaffolds. (G,H) Representative raft- and capview snapshots, respectively, from confocal fluorescence microscopy 3D renderings of tubular HUVEC tissue formations within Capgel. Green fluorescence: Actin Green 488 nm cytoskeletal staining; blue fluorescence: NucBlue nuclei staining. Black arrows indicate fully formed tubular vessels. Scale bars=50 μm.

FIG. 5. HDFs and HUVECs comprising microvessel tissues engineered with Capgel biomaterials align preferentially in the direction of the scaffold capillary long axis during four (4) weeks in culture. (A) Representative confocal fluorescence maximum z-projection micrograph showing NucBluestained nuclei of HDFs cultured in Capgel. (B) Overlay image of representative confocal fluorescence maximum z-projection and corresponding DIC micrographs of scaffold-cultured HDFs stained with NucBlue. (C) Polar plot quantifying the nuclear alignment of HDFs cultured in Capgel as determined by first fitting ellipses to binarized images (n=12) of NucBlue-stained nuclei and then measuring the angle of each best-fit ellipse major axis relative to the scaffold capillary long axis defined as either 0° or 180°. The upper half of the polar plot ranging from −90° to 90°, which includes 0°, describes nuclei in the top half of the analyzed images, while the lower half of the plot from 90° to 270°, which includes 180°, describes the nuclei in the bottom half of these images. The radial value of the arcs for blue-shaded sectors indicates the total number of nuclei, i.e., cells, within the covered range of relative angles. (D-F) These panels are the same types of images and plots as panels (A-C), respectively, but for HUVECs scaffold-cultured in Capgel (n=6 for (F)). Blue fluorescence: NucBlue nuclei staining; gray: DIC. Scale bars=50 μm for (A,D), 20 μm (B,E).

FIG. 6. Female Aedes aegypti mosquitoes swarm warmed BITES tissue loaded with blood and engage in frenzied blood meal acquisition behaviors. (A) Representative image captured from a video presenting a warmed (34-37° C.), blood-loaded BITES construct (cellularized with HDFs) in the raftview orientation to 50 mosquitoes for 15 min. (B) Image still taken from video recordings capturing the taking of a blood meal event by an Ae. aegypti female from a warmed, blood-loaded, non-cellularized Capgel block oriented in raftview. (C) Box plot summarizing the percentages of Ae. aegypti (out of 50 total) that demonstrated blood meal acquisition behaviors on BITES human dermal microvascular bed model tissues (HDF-cellularized) presented for either 4, 15, or 20 min in separate experiments. (D) Plot of abdomen width ratio (Equation (1)) increases over acquisition time for six (6) individual females taking blood meals from an HDF BITES construct presented for 15 min. The average abdomen width ratio at the average time to blood meal desistance for all mosquitoes analyzed is indicated by the blue sphere in the plot, with error bars for both values. Numbers listed next to each final width ratio point indicate the final estimates of blood volumes in the corresponding mosquito midguts, calculated as before (Equations (2) and (3)). In panel (A), the white asterisk indicates the HDF-cellularized BITES tissue loaded with blood; the black asterisk indicates polystyrene foam insulation. In the box plot (B), vertical brackets=whiskers within Tukey's 1.5×interquartile (IQR) range, gray boxes=IQR, stars=outliers, horizontal lines=medians, dots=means; note that the median for 4 min equals/overlaps Q3 and the whiskers for this presentation time fall within the IQR and therefore are not shown. Error bars in (C)=±standard deviation (SD). Scale bar=2 mm.

FIG. 7. Female Ae. aegypti penetrate through multiple layers of warmed, blood-loaded HDF BITES microvessel tissue structures with the fascicle of stylet mouthparts to take a blood meal and the tissues can be cultured for days post-blood-meal (PBM). (A) A stereomicrograph of a mosquito snap-frozen with liquid nitrogen during blood meal acquisition from an HDF BITES tissue loaded with blood. The fascicle filled with blood can be seen penetrating the right side of the tissue in the raftview orientation, with the labium characteristically bent and pulled away. (B,C) Representative DIC micrographs of HDF BITES microvessels filled with blood imaged in the raftview and capview orientations, respectively. RBCs confined within microvessels and that have flowed out of the ends are observable as multiple small, dark, circular/spherical particles in the images. (D-I) Representative series of DIC micrographs at different focal planes showing the mosquito fascicle/stylet mouthparts penetrating HDF BITES tissue (A) imaged in in situ, capview, and raftview orientation, respectively. (J-L) Representative DIC micrographs at different focal planes and increased magnification relative to (G-I), showing a zoomed-in view of the end of the stylet mouthparts within the HDF BITES tissue. (M-O) Representative confocal fluorescence maximum z-projection micrographs of HDF BITES tissues imaged in the raftview orientation immediately following presentation and blood meal acquisition by Ae. aegypti (0 min PBM), or after additional short (20 min) or longer (3 days) PBM culture times. Green dashed lines outline “interaction volumes” of the stylet mouthparts within HDF BITES tissue. Green fluorescence: Actin Green 488 nm cytoskeletal staining; blue fluorescence: Nucblue nuclei staining; gray: DIC. Scale bars=1 mm for (A), 50 μm for (B,C, J-L), and 100 μm (D-I, M-O).

FIG. 8 presents the graphical abstract of BITES.

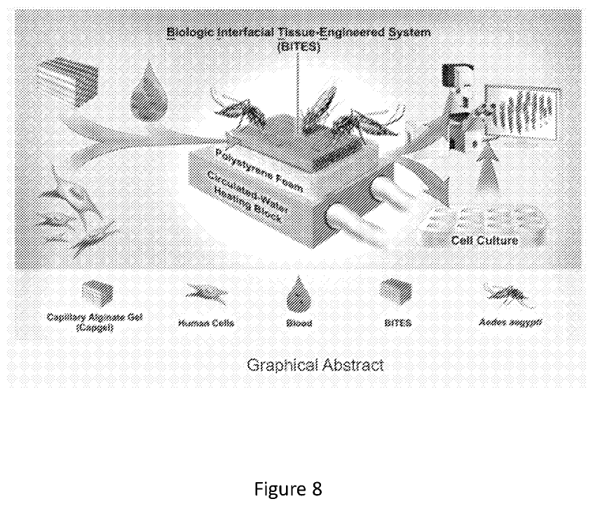

FIG. 9. (A) a blood-loaded Capgel for BITES on a heating block., and (B) a setting up of a video recorder and lighting equipment.

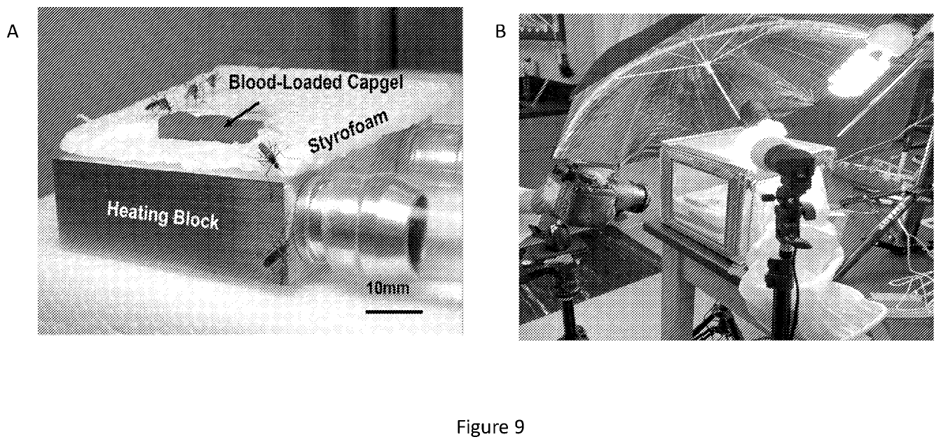

### DETAILED DESCRIPTION

### Definitions

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, the terms “a,” “an,” “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

As used herein, “about” refers to a value that falls within a 10-30% variance of the stated value. For example, an about 50% alginate solution refers to 50% alginate or a range of 35-65% alginate solution.

As used herein, “Capgel” refers to capillary alginate gel. Alginate is extracted as sodium alginates from brown seaweed, and alginate gel is commonly used as a binding, stabilizing and/or thickening additive gel due to their biocompatibility, nontoxicity, biodegradability, low-cost, and being simple to produce, and particularly valued for its application in foods and cosmetics [ISP Alginates, Section 3. Algin-Manufacture and Structure, in Alginates. Products for Scientific Water Control, 2000, International Specialty Products: San Diego, pp. 4-7]. Clinically, alginate is used in dental impression materials and hemostatic wound dressings [Blair, S. D. et al., Brit. J. Surg., 1990, 77 (5): 568-570; Rives, J. M. et al., Wounds—a Compendium of Clinical Research and Practice, 1997, 9 (6): 199-205].

Alginate gel is a linear polysaccharide of polymer chain, i.e., a linear copolymer with homopolymeric blocks of (1→4)-linked β-D-mannuronate (M) and α-L-guluronate (G) residues, respectively, covalently linked together in different sequences or blocks. The monomers may appear in homopolymeric blocks of consecutive G-residues (G-blocks), consecutive M-residues (M-blocks) or alternating M and G-residues (MG-blocks). Compositional variation is a reflection of source and processing. The pKa's of the C5 epimers are 3.38 and 3.65 for M and G respectively with the pKa of an entire alginate molecule somewhere in between [Schuberth, R. Ionotropic Copper Alginates. Investigations into the formation of capillary gels and filtering properties of the primary membrane, 1992, University of Regensburg: Regensburg; ISP Alginates, Section 3. Algin Manufacture and Structure, in Alginates. Products for Scientific Water Control, 2000, International Specialty Products: San Diego, pp. 4-7]. Alginate forms colloidal gels (high water content gels, hydrogels) with divalent cations such as Cd²⁺>Ba²⁺>Cu²⁺>Ca²⁺>Ni²⁺>Co²⁺>Mn²⁺, and among them Ca²⁺ is the best characterized and most used to form gels [Ouwerx, C. et al., Polymer Gels and Networks, 1998, 6 (5): 393-408].

As used herein, the term “copper capillary’ or “copper capillaries’ refers to the continuous parallel capillaries formed in the copper capillary alginate gels (CCAG) by allowing solutions of Cu²⁺ to diffuse uniformly into viscous solutions of alginate. These capillaries exhibit curved inner surfaces useful for seeding and propagating cells. The cross-section of the capillaries may be circular or non-circular. [Schuberth, R., Ionotropic Copper Alginates. Investigations into the formation of capillary gels and filtering properties of the primary membrane. 1992: Thiele, H., Histolyse und Histogenese, Gewebe und ionotrope Gele, Prinzipeiner Stukturbildung. 1967]. However, in common tissue culture media, CCAG alone swells, loses mechanical properties, and eventually dissolve due to a loss of copper ions that are released into the surrounding fluid environment. Accordingly, there is a need for a modified CCAG that provides a stable tissue scaffold in a cell culture environment or within a human or animal.

As used herein, the term “stabilizing agent” refers to a compound, ion, or moiety that reacts with the CCAG so that the resulting stabilized CCAG maintains its mechanical properties in a cell culture or within a human or animal and the stabilized CCAG is non-toxic to its surrounding environments.

As used herein, the term “carbodiimide chemistry” refers to carbodiimide crosslinker chemistry. Carbodiimide conjugation works by activating carboxyl groups for direct reaction with primary amines via amide bond formation. For example, EDC (1-ethyl-3-(-3-dimethylaminopropyl) carbodiimide), in conjunction with NHS (N-hydroxysuccinimide) allows 2-step coupling of two proteins without affecting the carboxyls of the second protein. First, EDC activates carboxyl groups and forms an amine reactive O-acylisourea intermediate that spontaneously reacts with primary amines to form an amide bond and an isourea by-product.

As used herein, the term “platform” refers to a standing structure made of biomaterials for testing various cells and chemical or biological substances on/in/within the structure.

As used herein, the term “scaffolds” or “bio-scaffolds” refers to three-dimensional (3D) porous, fibrous or permeable biomaterials intended to permit transport of body liquids and gases, promote cell interaction, viability and extracellular matrix (ECM) deposition with minimum inflammation and toxicity while bio-degrading at a certain controlled rate. Biomaterials such as collagen, various proteoglycans, alginate-based substrates and chitosan have all been used in the production of scaffolds for tissue engineering. Unlike synthetic polymer-based scaffolds, natural polymers are biologically active and typically promote excellent cell adhesion and growth.

As used herein, the term “cellularization’ or grammatical variations thereof, is intended to denote the cultivation and/or maintenance of cells in vitro, in particular in a small capillary or channel structure of biomaterials so as to allow them to partially colonize on the surface thereof or cover almost the whole surface thereof.

The term “3D bioprinting” means producing three dimensional objects from biomaterials by using 3D printing technology. An example of a bioprinting technique comprises

As used herein “biopolymer” refers to a polymer to which cells adhere and thrive. A non-limiting list of biopolymers includes alginate, gelatin, laminin, fibronectin or other extracellular matrix molecules, silicones, hyaluronic acid, methacrylate hydrogels, degradable polyester biomaterials (eg., PLA, PGA, PLLA, etc.) collagens, glycosaminoglycans, chondroitin sulfate, dermatan sulfate, and proteoglycans, chitosan and the like, or combinations thereof.

The term “microvessels” as used herein refers to tubular structures or channels through which liquid can flow. Microvessels may anatomically mimic microvasculature such as capillaries or small vasculature.

The term anatomically mimic refers to a shape, configuration and or size of an anatomic structure, or portion thereof. In certain examples herein where the bioscaffold includes microvessels, these would mimic vessels of a size or configuration to allow flow of a liquid similar to microvasculature.

### Overview

The implementation of bioengineering and tissue engineering approaches to the study of vector arthropod control and bite-site biology is a nascent area of investigation [22, 24, 35, 36]. The great promise of this developing area is clear and includes engineered avascular human skin equivalents [24], silicone microfluidic chambers for the direct collection and high-throughput downstream analysis of mosquito saliva [36], and acellular 3D-printed model skin composed of poly(ethylene glycol) diacrylate (PEGDA) and gelatin methacrylate (GelMA) biomaterials that include circulating blood for mosquito feeding [22].

### Mosquito Bites

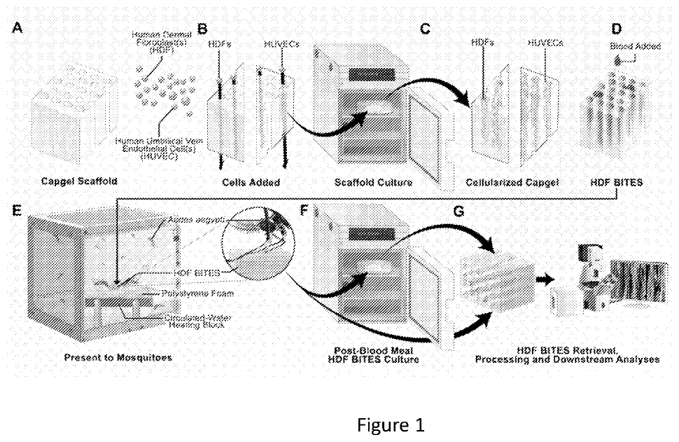

Detailed herein are proof-of-concept studies of 3D human dermal microvessel bed tissue models engineered with Capgel biomaterial scaffolds that female Ae. Aegypti mosquitoes bit, probed, and blood-fed from naturally. Here, this new in vitro platform for the study of arthropod bite-site biology is termed a Biologic Interfacial Tissue-Engineered System (BITES, FIG. 1).

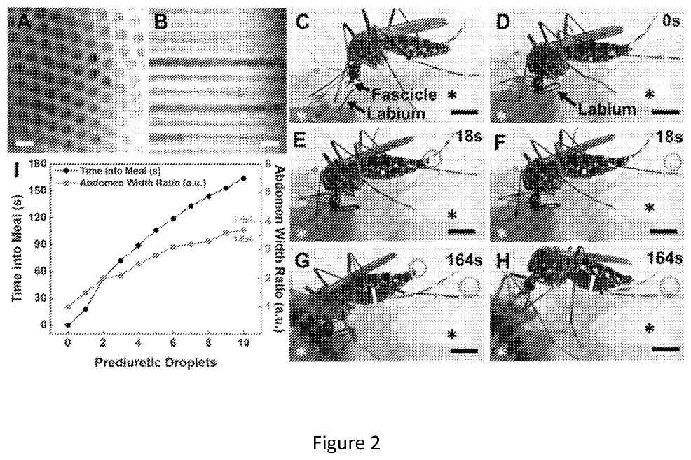

Blood meal acquisition by Ae. aegypti from warmed (34-37° C.), blood-loaded Capgel was documented with videography and examined. The defibrinated bovine blood was easily loaded into the Capgel and capable of freely flowing in the scaffold capillaries (FIG. 2A, B), which is required for uptake by the mosquito. In nature, mosquitoes generally engage in foraging behavior when seeking host blood meals [37]. After a host is found, Ae. aegypti initiate the blood-feeding process by first biting/penetrating the skin with the fascicle containing the stylet mouthparts and then probing the dermis for a blood microvessel(s) [38,39]. Once a suitable microvessel(s) is identified, the stylet mouthparts are fully inserted and the labium (i.e., sheath) part of the proboscis pulls away from the stylet and remains outside the host skin [39,40]. All these behaviors were clearly demonstrated by female Ae. aegypti during blood meal acquisition from the blood-loaded, warmed Capgel in this study (FIGS. 2C, D).

A mosquito starts expectorating saliva from the hypopharynx stylet mouthpart as soon as the skin is penetrated by the fascicle to act as a lubricant, inhibit coagulation, and modulate host immune responses [40-42]. The female then begins to ingest blood through a combined structure similar to a straw, formed by the hypopharynx and labrum stylet mouthparts [38]. This blood then fills the midgut, which is accompanied by an increase in abdominal width [40]. Though the expectoration of saliva was not directly observed from mosquitoes engaging in blood meal acquisition behaviors from Capgel, blood filling the midgut and associated abdominal expansion was seen, and these processes—saliva expectoration and blood ingestion—are inextricably linked biologically (FIGS. 2D-I).

In a given meal, a mosquito generally ingests more weight in blood than its total body weight [43], and there is a possibility of midgut rupture if more than ˜5-7 μL of blood is ingested [40]. To avoid rupture, mosquitoes engage in a prediuretic process to concomitantly excrete 10 or more droplets of urine during an average blood meal, to concentrate the erythrocyte content (nutrients) in the midgut and effectively increase the total protein intake [44,45]. Additionally, prediuresis cools the mosquito and keeps the body temperature within safe physiological limits [46]. Blood-feeding times for Ae. aegypti range from 32.7 to 307.6 s according to Ribeiro et al. [34], and Stobbart reports 2.50±0.66 μL for the mean (+SD) repletion volume (not corrected for prediuresis) [47].

In the studies explained in further detail in the Examples, the Ae. aegypti spent 163 s taking a blood meal from the warmed, blood-loaded Capgel, excreted 10 prediuretic drops during that time, had a final abdomen width ratio of 3.9, and ingested a calculated estimate of 2.0 μL of blood in total, i.e., including the estimated 0.2 μL of prediuretic excretions (FIG. 2). Altogether, these results strongly support the assertion that female Ae. aegypti mosquitoes acquired blood meals from warmed, blood-loaded Capgel in a manner mimicking the typical natural behaviors exhibited during the taking of a blood meal from vertebrate hosts.

Capgel Platform BITES Cellularized with Human Microvascular Cells

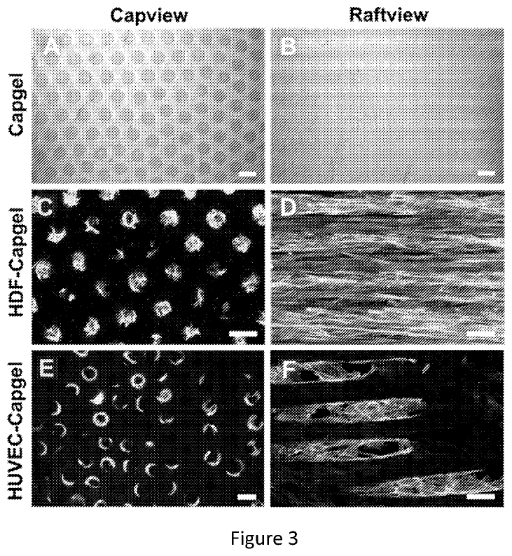

The unique uniform capillary microstructure of Capgel biomaterials provides an excellent tissue scaffold to engineer stylized dermal microvessel beds (FIG. 3A,B) with capillary channel diameters between ˜50 and 70 μm, approximating those of the dermal microvasculature once cellularized [48]. Fibroblasts and endothelial cells (here, specifically HDFs and HUVECs) were chosen for these proof-of-concept experiments because both cell types are integral components of microvessels, and the dermis is populated in part by fibroblasts [49-53]. Further, emphasis was placed on modeling the dermis/dermal microvessels because this tissue is the primary target for the bites of hematophagous arthropods such as mosquitoes.

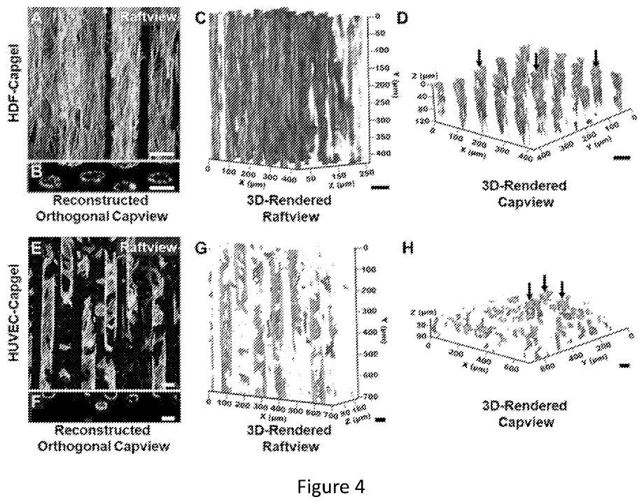

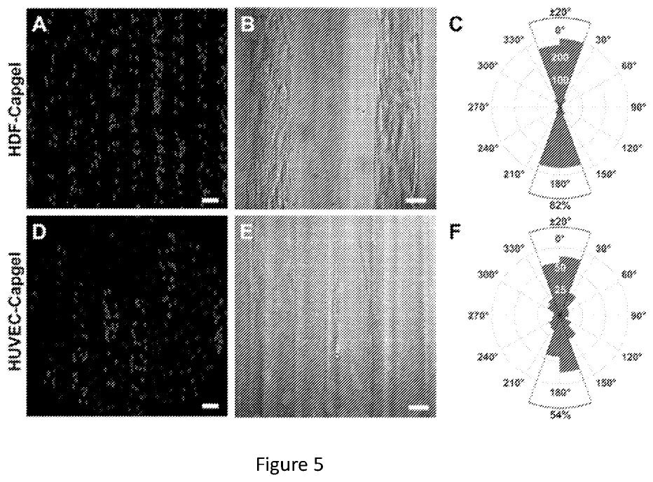

Both HDFs and HUVECs colonized the Capgel scaffolds and lined the capillary channels with patent, 3D tubular, microvessel-like tissues composed of oriented cells (FIGS. 3C-F, 4 and 5). The HDF microvessel structures were longer and contiguous within the scaffolds, compared with the patchy HUVEC microvessel tissues (FIG. 4). In natural vessels, cells are preferentially orientated in the direction of blood flow, and this alignment reflects and facilitates the structure and function [54-57]. Analogously, the majority—82% and 54% of HDFs and HUVECs, respectively—of cells cultured in Capgel were also aligned (±20°) in the direction of the scaffold capillary long axis (FIG. 5). These alignment behaviors likely stem from both the inherent capacities of the cells to orient in response to scaffold features and the configuration of the Capgel scaffolds. Similar alignment phenomena have been documented previously for fibroblasts and endothelial cells (60% and 50%, respectively) cultured in confined, 50-μm-wide, gelatin methacrylate hydrogels [58].

Overall, these results suggest that the scaffold culture conditions were sufficient for HDFs but may need to be adjusted for HUVECs. Such adjustments could include (1) an increased initial HUVEC seeding density, (2) longer culture periods, and/or (3) co-culture in scaffolds first lined with HDF structures. The co-culture approach is particularly attractive and will be a focus of future BITES work as it is known that these cell types cooperate to form robust tubular endothelial structures and this arrangement mimics the natural microanatomy [49-52].

Evaluation of BITES with Mosquitoes

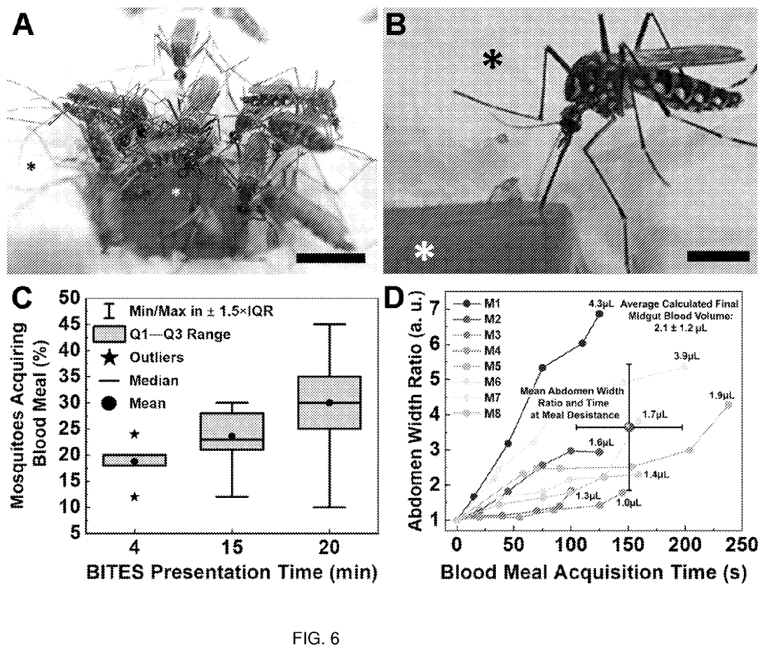

Once the BITES constructs were developed, experiments were conducted to evaluate the acquisition of blood meals by female Ae. aegypti mosquitoes from these engineered tissues. Only BITES tissue cellularized with HDFs was advanced into these proof-of-concept experiments because it was the best model representation of fully cellularized beds of microvessel tissue structures engineered with human cells. Mosquitoes swarmed the HDF BITES that was warmed and loaded with blood (FIG. 6A), indicating that they were attracted to the tissue. The percentage of Ae. aegypti that engaged the engineered tissue was similar for different presentation times (FIG. 6B), which suggests that these constructs need only be presented to mosquitoes for tens of minutes at most to conduct effective experiments. On average, an individual female took 151±46 s to acquire a blood meal (roughly in the middle of Ribeiro et al.'s range [34]), and the relative amount of blood ingested quantified by the nondimensionalized abdomen width ratio metric was 3.6±1.8, and the calculated estimate of the blood volume in the midgut was 2.1±1.2 μL (FIG. 6C). The largest observed ratio was almost seven (7), which corresponded to 4.3 μL of blood (calculated estimate) in the midgut of this mosquito, and 37.5% of the analyzed Ae. aegypti fed near or to repletion, with calculated midgut blood volumes of 1.9 μL or above (i.e., within 0.66 μL—one SD—of 2.5 μL [47]).

All these data together strongly support the assessment that blood meal acquisitions by female Ae. aegypti mosquitoes from warmed, blood-loaded HDF BITES microvessel tissue beds mimic natural acquisitions from vertebrate hosts.

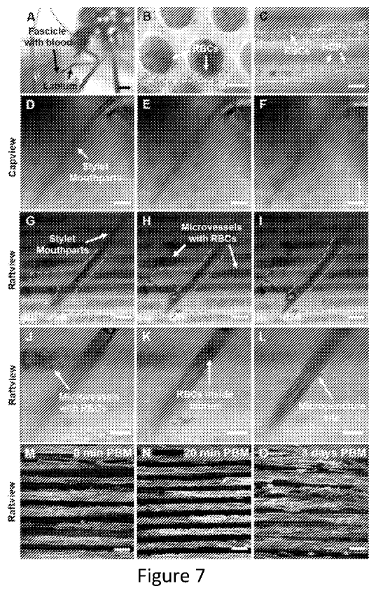

As with the blood-loaded Capgel, HDF cellularization did not prevent the free movement/flow of blood within the BITES microvessels (FIG. 7B,C). A DIC micrograph and corresponding 3D-rendered depth profiling of the situation revealed that the mosquito pushed the fascicles of stylet mouthparts ˜500 μm into the BITES tissue, a depth at which dermal microvessels would be encountered in humans (Figure S3) [48,59]. A closer inspection of this penetration by DIC microscopy in the capview and raftview orientations showed that several layers of the microvessel structures were pierced (FIG. 7D-I, respectively) and a track with an irregular “interaction volume” was created (FIG. 7G-I, green dashed lines). The paucity of RBCs observed in this track and interaction volume indicates that the total volume of the blood meal was extracted from multiple BITES microvessel structures, even with the apparent narrowness of the punctures (FIG. 7J-L).

Confocal microscopy additionally demonstrated that the BITES microvessel structures were intact after blood meal acquisitions and that BITES tissue can be viably cultured for at least three days following presentation to and engagement by mosquitoes (FIG. 7M-O).

### Further Uses of Cellularized BITES

No signs of apparent contamination were observed in these post-blood-meal BITES tissue cultures. Antibiotics (penicillin and streptomycin) were standard components of all culture media in this study, helping to reduce/abrogate the bacterial load. The use of these compounds in BITES studies with other potential vectors (e.g., ticks) and pathogens such as Borrelia burgdorferi will likely require careful consideration of both the antibiotic dose and class used, if any. Similar considerations may also be important if microbiome studies are envisioned. These data, especially the demonstration that BITES tissue can be cleanly cultured post-blood-meal for some days, highlight the potential application of the platform in future studies investigating phenomena such as vectored pathogen infection sequences, kinetics, and sequelae.

Disease outcomes for a host bitten by an infected arthropod are closely linked to the early events that occur during the bite, such as host immune modulation and pathogen replication/enhancement at the skin bite site [9]. These events play a crucial role in the development of a disseminated infection within the host and the continuation of the pathogen transmission cycle [6]. The interplay between the biting arthropod, its expectorated saliva, the transmitted pathogens, and the host cells at the skin bite site is essential to understanding these complex interactions [6,60-63]. Gaining this understanding is vital for the development of effective therapies and preventative strategies against arthropod-borne pathogens and diseases. Numerous studies involving various viruses and mosquito species have shown that, along with the pathogen's strategies to overcome the host immune response, the expectorated saliva also has a critical role in the initial pathogen replication process [6,60-63]. However, live animal models often use artificial infection, such as intravenously or intraperitoneally, which fails to accurately replicate natural vector-bite mediated pathogen delivery [64,65]. Even if the pathogens are injected intradermally, this delivery method does not fully replicate delivery by natural biting/blood-feeding [66].

It is believed that there has been no system which incorporates any cell of the skin tissue (resident or migratory) and therefore does not currently offer the potential to investigate vector-host-pathogen dynamics at the cellular or molecular levels.

The BITES platform presented in this study possessed microvessel structures with small diameters that closely approximate the sizes of microvessels found at depths in the dermis that mosquitoes access for blood-feeding [48,59,67]. Further, these BITES microvessel structures resulted from Capgel self-assembly, thus not requiring 3D printing, and were cellularized.

In addition, since animals, including companion animals, farm animals, zoo animals, wild animals, and non-human primates are also infected by virus or parasites through mosquitoes- or other insect bites, including ticks, fleas, and flies, etc., blood extracted from those animals can be used in this Capgel platform with those animal originated endothelial cells, fibroblasts, keratinocytes and/or other cells found in their epidermis and/or dermis in order to develop a new veterinary medicines to prevent Eastern equine encephalitis, Western equine encephalitis, Venezuelan equine encephalitis, Japanese encephalitis, West Nile virus and fowl pox and/or other insect borne animal diseases as well as in order to develop a new approach to human insect borne diseases such as Chikungunya (CHIKV), Crimean Congo haemorrhagic fever (CCHF), Dengue, yellow fever, leishmaniasis, Lyme disease, malaria. tick-borne encephalitis (TBE), West Nile virus (WNV), and the like. BITES model can also be applied to testing of the effectiveness of various substances for their use as repellent or insecticide.

### SUMMARY OF EXPERIMENTAL RESULTS

Vector-borne diseases transmitted through the bites of hematophagous arthropods, such as mosquitoes, continue to be a significant threat to human health globally. Transmission of disease by biting arthropod vectors includes interactions between (1) saliva expectorated by a vector during blood meal acquisition from a human host, (2) the transmitted vector-borne pathogens, and (3) host cells present at the skin bite site. Currently, the investigation of bite-site biology is challenged by the lack of model 3D human skin tissues for in vitro analyses.

To help fill this gap, a tissue engineering approach is employed here to develop new stylized human dermal microvascular bed tissue approximates-complete with warm blood-built with 3D capillary alginate gel (Capgel) biomaterial scaffolds. These engineered tissues, termed a Biologic Interfacial Tissue-Engineered System (BITES), were cellularized with either human dermal fibroblasts (HDFs) or human umbilical vein endothelial cells (HUVECs). Both cell types formed tubular microvessel-like tissue structures of oriented cells (82% and 54% for HDFs and HUVECs, respectively) lining the unique Capgel parallel capillary microstructures.

Female Aedes (Ae.) aegypti mosquitoes, a prototypic hematophagous biting vector arthropod, swarmed, bit, and probed blood-loaded HDF BITES microvessel bed tissues that were warmed (34-37° C.), acquiring blood meals in 151±46 s on average, with some ingesting &4 μL or more of blood. Further, these tissue-engineered constructs could be cultured for at least three (3) days following blood meal acquisitions. Altogether, these studies serve as a powerful proof-of-concept demonstration of the innovative BITES platform and indicate its potential for the future investigation of arthropod bite-site cellular and molecular biology.

### Exemplary Embodiments

In this disclosure, it is demonstrated that Capgel for BITES is an effective platform to study the behavior of mosquitoes. Capgel for BITES is a scaffold of densely-packed parallel micro-capillary structure, which is an elastomeric hydrogel made of alginate and gelatin that forms a stable matrix of peptide-bond crosslinked network of alginate polysaccharide chains and gelatin polypeptide chains.

The primary component of these hydrogels is alginate, a popular natural anionic linear polysaccharide biopolymer composed of β-D-mannuronic and α-L-guluronic acids residues. Initial ionic crosslinking of an alginate solution via uniaxial diffusion of divalent metal ions such as Cu²⁺ generates the Capgel self-assembled micro-capillary structure. Capillary diameter and density can be tailored for a given application via selection of the initial alginate and/or diffusing divalent metal ion (i.e., Cu²⁺) concentrations [Axpe, E. and M. L. Oyen, Int J Mol Sci, 2016, 17 (12): 1976; Lee, K. Y. and D. J. Mooney, Prog Polym Sci, 2012, 37 (1): 106-126].

Since alginate does not have cell attachment sites, gelatin, which has intrinsic Arg-Gly-Asp (RGD) cell-adhesion motifs, is added to the initial alginate solution [Gungor-Ozkerim, P. S. et al, Biomater Sci, 2018, 6 (5): 915-946; Neufurth, M. et al., Biomaterials, 2014, 35 (31): 8810-8819; Zhang, T., K. C. Yan, L. Biofabrication, 2013, 5 (4): 045010]. After ionic crosslinking, these gels are sectioned into blocks, subjected to carbodiimide chemistry to form peptide crosslinks to produce Capgel scaffolds.

Briefly, in a non-limiting example, the Capgel for BITES is manufactured by the method comprising steps of:
a. dissolving biopolymer, optionally gelatin, in distilled and deionized water;
b. degrading the biopolymer solution and equilibrate the solution to room temperature;
c. adding alginate to the biopolymer solution to form an alginate/biopolymer solution;
d. disposing the alginate/biopolymer solution in a container coated with dehydrated alginate;
e. covering the container with a porous cover layer soaked with copper (II) sulfate solution;
f. applying copper sulfate solution dropwise to the alginate/biopolymer solution, optionally through the porous cover layer;
g. allowing the alginate/biopolymer solution to set into a capillary alginate gel (Capgel) comprising capillaries;
h. cutting the Capgel into segments;
i. optionally, crosslinking the Capgel segments via carbodiimide chemistry.

Further, the capillaries of the Capgel for BITES can be cellularized by steps of
a. dissociating about 80% confluent cells in the cell culture container with trypsin;
b. adding serum-containing cell culture medium to the cell culture container having trypsinized cells;
c. moving the contents of the cell culture container into a tube for centrifugation;
d. discarding supernatant after centrifugation to get a cell pellet;
e. adding a small volume of the cell culture medium to the cell pellet and suspending the pellet;
f. calculating cell number; and
g. applying the cells to the capillaries of the Capgel;
h. placing the Capgel with capillaries in the vertical direction in a microcentrifuge tube and briefly centrifuging the tube;
i. placing the cell-seeded Capgels into the incubator to allow cell attachment;
j. repeating steps of g, h, and i two times or more times so that enough number of cells are seeded per Capgel block;
k. adding the cell culture medium by submerging Capgel blocks; and
l. culturing the cells within the Capgel blocks;
wherein after cell culture, optionally the cell culture medium can be replaced with host animal's serum or blood for mosquito behavior observation.

Although alginate and gelatin are used to make Capgel in this disclosure, other biopolymers can be contemplated for the Capgel, and the biopolymer can be at least one selected from a group comprising alginate, gelatin. collagen, hyaluronic acid, chitosan, laminin, fibronectin, glycosaminoglycans, chondroitin sulfate, dermatan sulfate, proteoglycans or other extracellular matrix molecules, silicones, methacrylate hydrogels, degradable polyester biomaterials (e.g., PLA, PGA, PLLA, etc.) the like, and combination thereof.

In preferred embodiments, the capillaries are cellularized with a plurality of cells, wherein the cell type is at least one selected from dermal fibroblasts, vascular endothelial cells, keratinocytes, immune cells, and mesenchymal stem cells, wherein immune cells optionally comprise macrophages, either tissue-resident or derived from infiltration of monocytes, or dendritic cells. In some embodiments, the microtubular capillaries are cellularized with HDFs and/or HUVECs alone or with other cell types, optionally dermal keratinocytes and/or macrophages.

In certain embodiments, the diameters and lengths of each of the microtubular capillaries may be the same, similar or vary. In certain embodiments, the diameter of a microtubular capillary may be the same, similar or vary between one end and the other end of the capillary. In certain embodiments, the cross-section of each of the microtubular capillaries is circular or non-circular. In certain embodiments, some of the microtubular capillaries are open at both ends and the others are open at one end. In certain embodiments, the diameter of each of the microtubular capillaries is between about 10 μm to about 300 μm, in particular between about 50 and 70 μm. The density of the capillaries and the average diameter of the capillaries can be controlled by varying the ratio of alginate, gelatin, and cupper.

Then, to use the cellularized Capgel for BITES for use as a mosquito bite model, the following steps can be performed;
a. removing the cell culture medium from the capillaries of the Capgel for BITES;
b. adding human body fluid, in particular human blood into the capillaries; and
c. putting the Capgel for BITES on a 37° C. heating block.

In certain embodiments, the microtubular capillaries are loaded with cell culture medium, serum and/or blood, and optionally the capillaries having open ends at both sides can be connected to a peristaltic pump or height-adjusted syringes to make the cell culture medium, serum and/or blood flow through the microtubular capillaries.

In certain embodiments, the surface of Capgel can be coated with a substance such as insecticide or repellant to be tested and the CapGel is subjected to one or more arthropods for observing and/or video-recording behavior of the one or more arthropods.

In other embodiments, the microtubular capillaries, either independently, or in addition to the cellularization process, may be loaded with an alcohol (e.g. propylene glycol), sugar, sugar alcohols (e.g. erthritol) and/or insecticides, or solutions of any thereof. Sugars used for loading the capillaries include, but are not limited to, sucrose, glucose, fructose, and the like. Accordingly, in one example, step (k) of paragraph 0082 above can be modified to replace cell culture medium with an alcohol, sugar, sugar alcohol or insecticide, or alternatively the process of cellularization can further involve loading the microtubular capillaries with an alcohol, sugar, sugar alcohol or insecticide. In an alternative embodiment, the BITES model is not cellularized but still loaded with an alcohol, sugar, sugar alcohol or insecticide. For example, the process of paragraph 0081 can further include the step of loading such substances into the microtubular capillaries.

### 3D Printing Technologies

According to known methods of 3D printing construction of a three-dimensional structure is typically performed in a step-wise manner, typically through one or more needles, layer by layer and includes for example extrusion, direct energy deposition, solidification of powder, photopolymerisation and sheet lamination. In particular, layer formation is performed through solidification of photo curable resin under the action of visible or UV light irradiation. Alternatively, the three-dimensional structure can be created continuously from a liquid interface (see for example WO2014126837 or U.S. Pat. No. 7,892,474). All these 3D printing methods rely on the properties of the 3D printing composition to define the microstructural parameters and possibly biochemical properties of the printed structure. Hence, the properties of a 3D printed structure are limited not only by the capability of the printing method, but also by the printing composition used. One specific example of a method of bioprinting biomaterial is set forth in Panarello et al., Gels 2022, 8:376.

Stereolithography. The technique builds three-dimensional models from liquid photosensitive polymers that solidify when exposed to ultraviolet light. According to this technique, the model is conventionally built upon a platform situated just below the surface in a vat of liquid epoxy or acrylate resin. A low-power highly focused UV laser traces out the first layer, solidifying the model's cross section while leaving excess areas liquid. Next, an elevator incrementally lowers the platform into the liquid polymer. A sweeper re-coats the solidified layer with liquid, and the laser traces the second layer atop the first. This process is repeated until the prototype is complete. Afterwards, the solid part is removed from the vat and rinsed clean of excess liquid. Supports are broken off and the model is then placed in an ultraviolet oven for complete curing. As discussed below, stereolithography is the technique which is most easily adaptable to product of anatomical model embodiments as taught herein. These models could be constructed using standard stereolithography equipment by substituting hydrogel slurries for the materials normally employed in the resin bath. These hydrogel slurries may comprise alginate, gelatin. Laminin, fibronectin or other extracellular matrix molecules, silicones, hyaluronic acid, methacrylate hydrogels, degradable polyester biomaterials (e.g., PLA, PGA, PLLA, etc.) Collagens, glycosaminoglycans, chondroitin sulfate, dermatan sulfate, and proteoglycans, chitosan and the like.

Ink-Jet printing. Ink-Jet Printing refers to an entire class of machines that employ ink-jet technology. The first was 3D Printing (3DP), developed at MIT and licensed to Soligen Corporation, Extrude Hone, and others. The ZCorp 3D printer, produced by Z Corporation of Burlington, MA (www.zcorp.com) is an example of this technology. Parts are built upon a platform situated in a bin full of powder material. An ink-jet printing head selectively deposits or “prints” a binder fluid to fuse the powder together in the desired areas. Unbound powder remains to support the part. The platform is lowered, more powder added and leveled, and the process repeated. When finished, the green part is then removed from the unbound powder, and excess unbound powder is blown off. Finished parts can be infiltrated with wax, CA glue, or other sealants to improve durability and surface finish. Typical layer thicknesses are on the order of 0.1 mm. 3D Systems' (www.3dsystems.com) version of the ink-jet based system is called the Thermo-Jet or Multi-Jet Printer. It uses a linear array of print heads to rapidly produce thermoplastic models.

Fused Deposition Modeling. In this technique, filaments of heated thermoplastic are extruded from a tip that moves in the x-y plane. Like a baker decorating a cake, the controlled extrusion head deposits very thin beads of material onto the build platform to form the first layer. The platform is maintained at a lower temperature, so that the thermoplastic quickly hardens. After the platform lowers, the extrusion head deposits a second layer upon the first. Supports are built along the way, fastened to the part either with a second, weaker material or with a perforated junction. Stratasys, of Eden Prairie, MN makes a variety of FDM machines ranging from fast concept modelers to slower, high-precision machines.

### EXAMPLES

### Example 1. Materials and Methods

### 1.1. Mosquito Rearing

Ae. aegypti mosquitoes were reared as reported previously [32]. Briefly, 8 mg of eggs were brushed off germination paper and were added into a glass vial with 7.5 mL of larval food composed of 3% (g/mL) liver powder (MP Biologics, Santa Ana, CA, USA) and 2% (g/mL) brewer's yeast (#1700, Insectrearing.com, Newark, DE, USA) in deionized (DI) water. Mosquito eggs and larval food were vigorously shaken and decanted into a tray of 3 L of DI water and incubated at 29-30° C.; this was considered day 0. On day 3, 7.5 mL of larval food was added to the larval rearing tray, followed by 10 mL on day 4 and day 5. On day 6, pouring/rinsing was performed over a 500 μm strainer and the larvae and pupae were transferred to a 50 cm² surface area cup with 200 mL of DI water. The cup was kept in a rearing cage (8×8″ Bioquip, Rancho Dominguez, CA, USA). Within 24 h after the rinse, the majority of the mosquitoes had emerged inside the cage. Finally, a cotton ball saturated with 10% sucrose (ThermoFisher, Waltham, MA, USA) was provided ad libitum as a food supply for the mature mosquitos by placing it on top of the rearing cage. On the day before experimentation, the sucrose cotton ball was removed and a cotton ball laden with DI water was provided ad libitum overnight.

### 1.2. Capgel Scaffold Synthesis and Processing

### (1) Formation and Growth of Capgel Hydrogels:

The synthesis of Capgel blocks was conducted as previously described [26]. Briefly, 10% bloom gelatin (G1890, Sigma-Aldrich, Saint Louis, MO, USA) was dissolved in distilled, deionized (ddH₂O) water and degraded by heating with sodium hydroxide (NaOH) at 80° C. for 72 h.

An oligomeric 10% gelatin solution was equilibrated to ˜23° C. for 2 h and mixed with a sodium alginate solution (Protanal Pharm Grade LF10/60, FMC Biopolymer, Philadelphia, PA, USA, supplied by IMCD US, LLC, Rochelle Park, NJ, USA) to create a 3% alginate, 2.6% gelatin solution.

A 10 cm glass petri dish was prepared by coating its surfaces with 4% alginate dehydrated at 80° C. The parent solution was then added to the alginate-coated dish and placed into a larger glass container. A 0.1 M copper (II) sulfate pentahydrate (CuSO₄ 5H₂O, Acros Organics, Flanders, Belgium) soaked Kimwipe was held taut by a plastic ring over the top of the petri dish, and an additional 0.1 M CuSO₄ was then added dropwise to the surface of the Kimwipe for 10 min. The Kimwipe was then removed, and the entire petri dish was submerged in 0.1 M CuSO₄ for approximately 72 h. After the parent gel had grown, it was cut into strips and rinsed extensively three times in ddH₂O over the following three days. The strips were then sectioned into ˜5×5×3 mm blocks.

### (2) Crosslinking and Preparation of Capgel Blocks:

Then, Capgel blocks were crosslinked via carbodiimide chemistry as previously described [26] . . . . Briefly, four ˜5×5×3 mm Capgel blocks were added in a 50 mL conical tube, which was then filled with 20 mL of PBS containing 1.89 mg/mL of N-hydroxysuccinimide (ThermoFisher, Waltham, MA, USA). The conical tube was shaken gently overnight at 4° C. Then, 20 mL of PBS containing 1.57 mg/mL N-(3-Dimethyl-aminopropyl)-N′-ethylcarbodiimide hydrochloride (Sigma-Aldrich, Saint Louis, MO, USA) was added to the conical tube, yielding a final reaction volume of 40 mL, which was gently shaken overnight at 4° C. After completion of the crosslinking reaction, Capgel blocks were washed extensively for three to four days, with 0.2 μm filtered (564-0020, Nalgene, Rochester, NY, USA), sterile saline solution (0.9% NaCl, S271-3, ThermoFisher, Waltham, MA, USA). The rinses were repeated with filter-sterilized 10× sodium citrate solution (BP1325-4, ThermoFisher, Waltham, MA, USA), followed by another three rinses in sterile saline. Capgel blocks were then autoclaved using a liquid cycle and sterilization hold of 15 min. Sterilized Capgel blocks were then stored at 4° C. in sealed glass bottles.

### 1.3. Capgel Scaffold Cellularization

Capgel blocks were transferred to a 6-well plate (CytoOne, Ocala, FL, USA) with a sterile spatula (Corning Inc., Corning, NY, USA), in groups of four per well. To culture human dermal fibroblasts (HDF, CRL-2522, ATCC, Manassas, VA, USA), 5 mL of Dulbecco's Modified Eagle Medium (DMEM, Gibco, ThermoFisher, Waltham, MA, USA) supplemented with 10% fetal bovine serum (FBS, Gibco, ThermoFisher, Waltham, MA, USA) and 1% penicillin-streptomycin (Gibco, ThermoFisher, Waltham, MA, USA) was used to submerge and culture HDF-Capgel blocks.

To culture human umbilical vein endothelial cells (HUVECs, C0035C, Gibco, ThermoFisher, Waltham, MA, USA), 5 mL of Human Large Vessel Endothelial Cell Basal Medium 200 (Gibco, ThermoFisher, Waltham, MA, USA) supplemented with Large Vessel Endothelial Supplement (LVES, Gibco, ThermoFisher, Waltham, MA, USA) and 1% penicillin-streptomycin was used to submerge and culture HUVEC-Capgel blocks.

The plate was then placed into a 37° C., 5% CO₂ incubator (ThermoFisher Scientific Forma Series II Model 3110, Waltham, MA, USA) overnight. HDF and HUVEC cell lines were cultured in T-175 Cell Culture Flasks (Corning Inc., Corning, NY, USA) containing 25 mL of respective cell culture media.

At 80% confluency, cells were incubated with 10 ml of 0.05% trypsin (Gibco, ThermoFisher, Waltham, MA, USA) for 5 min and spun down at 1905 relative centrifugal force (RCF) for 3 min (Thermo IEC Centra GP8R, 216 4-place swinging bucket rotor, ThermoFisher, Waltham, MA, USA) in a centrifuge.

After centrifugation, the supernatant was removed, and the concentration of the resulting cell pellet was calculated using a phase counting chamber (Hausser Scientific, Horsham, PA, USA) and diluted to 40,000 live cells/μL.

The media was then removed from the 6-well plate containing the Capgel blocks and a sterile gauze pad (4×4, 12-ply, ThermoFisher, Waltham, MA, USA) was used to remove excess media from the surface of the Capgel. One microliter of the cell pellet was then applied using a pipette tip to dispense and spread the cell suspension across the capillary openings of the Capgel. Capgels were placed with capillaries in the vertical direction in microcentrifuge tubes and briefly centrifuged. The cell-seeded Capgels were then placed into the incubator for 30 min to allow cell attachment.

This process of seeding and incubating was repeated two more times so that a total of 3 μL of the cell pellets was seeded per Capgel block (120,000 cells/Capgel). Then, the Capgels were incubated for an additional 2 h to allow cell attachment, before adding 5 mL of media to each well, submerging the blocks. The cells were then cultured within the Capgel blocks for 4 weeks, undergoing a media change every two to three days.

### 1.4. Videography

In a rearing cage with one optically clear side (8×8″ Bioquip, Rancho Dominguez, CA, USA), 20-50 Ae. aegypti female mosquitoes were sorted and sucrose-starved for ˜18 h. The cage was placed under filming lights for 45 min so that the mosquitoes became acclimated to the lights and the room temperature before feeding (Figure S1A). A camera (Olympus Tough camera with an Ultimax 40.5 mm macro lens) was set in front of the clear side of the cage to record the blood-feeding events. A water bath (Isotemp 4100 H5P, ThermoFisher, Waltham, MA, USA) circulated warm water through an aluminum heat sink to regulate the temperature of the blood-loaded Capgel/BITES at 37° C. The Capgel/BITES was placed on the heat sink immediately after blood loading in raftview orientation. Bare surfaces of the warm heat sink were covered with a thin piece of polystyrene foam insulation to localize heated areas to the Capgel/BITES and avoid diverting the mosquitos (Figure S1B).

### 1.5. Blood Meal Acquisition Experiments

After the mosquitoes were acclimatized to the videography setup, a Capgel block stored in saline solution at 4° C. or an HDF-cellularized Capgel block submerged in media at 37° C. was retrieved using a sterile spatula. The Capgel block was wiped on all sides with a sterile gauze pad for 30 s to remove excess saline/media and was placed in a petri dish. Using an inverted microscope (20×), the Capgel was placed on the petri dish with capillaries oriented in the vertical direction, and 10 μL of warm (37° C.) defibrinated bovine blood (HemoStat Laboratories, Inc., Dixon, CA, USA; the product was stored for less than two (2) weeks at 4° C. and used as received without modification) was slowly added on top of the capillaries, without dripping blood onto the external surfaces of the block. Blood was allowed to flow through the capillaries until erythrocytes were seen emerging from the other side of the Capgel using an inverted microscope. Next, with the sterile spatula, the Capgel was flipped 180° and 10 μL of blood was added on the opposite capillary ends.

This process was repeated once more to achieve a final loaded blood volume of 40 μL in the Capgel/BITES. With blood loaded in the capillaries, the Capgel/BITES was transferred with sterile plastic forceps onto a sterile gauze pad with its capillaries horizontally disposed and carefully wiped with gauze on sides without capillary openings.

The HDF-cellularized BITES was presented as described above at different time intervals (4, 15, and 20 min) while recording the percentage of mosquitoes (n=50) probing, biting, and/or blood-feeding every minute. To assess the blood meal duration and quantity of blood acquired, video recordings of mosquitoes (n=50) acquiring blood from BITES were analyzed to assess changes in the abdomen width across time. Frames from the video were analyzed in ImageJ v1.53 (National Institute of Health, Bethesda, MD, USA) to calculate initial, intermediate, and final abdomen lengths by measuring the pleural membrane from tergum to sternum at the fourth abdominal segment. Measurements were scaled relative to the diameter of the head (0.67 mm). An abdomen width ratio was determined by the following equation:

$$Abdomen Width Ratio = Wt / Wi \qquad ( 1 )$$

where Wt is the abdomen width (mm) at a given time(s) during blood-feeding and Wi is the abdomen width initially (i.e., prior to any ingestion/blood-feeding). To calculate the total volume of blood ingested, the volume of the abdomen was approximated as an ellipsoid, and mosquito abdominal dimensions were measured in ImageJ. The following equation was used to find the volume:

$$Ellipsoid Volume = ( 4 / 3 ) π abc \qquad ( 2 )$$

where a is half the abdomen length taken from the abdomen-thorax border to the distal tip of the anus, b is half the abdomen width taken from tergum to sternum, and c is half the back of the mosquito across the midline and perpendicular to the long axis of the abdomen. The individual mosquito abdomen volume difference before and after blood feeding was used to calculate the blood volume ingested:

$$Δ V = Vf - Vi \qquad ( 3 )$$

where Vf and Vi are the final and initial abdomen volumes, respectively, and ΔV is the calculated blood volume ingested (μL). The volume of prediuretic excretion (i.e., urine) was approximated to the volume of a sphere:

$$Sphere Volume = ( 4 / 3 ) π {r}^{3} \qquad ( 4 )$$

where r is the radius of the prediuretic droplet measured using ImageJ. The calculated volume was then multiplied by the number of prediuretic droplets observed to determine the total volume excreted.

For experiments capturing in situ fascicle penetration in the BITES, the blood-feeding protocol was followed as described above until a mosquito was seen to be engaged and acquiring a blood meal. With a mosquito stably engaged with the BITES, liquid nitrogen was poured over the mosquito-BITES to freeze the stylet mouthparts inside the scaffold.

The BITES with the mosquito with the inserted stylet was then carefully placed in a petri dish and fixed immediately with 4% PFA, followed by permeabilization and staining (Section 2.7). The sample was imaged (Section 2.8) immediately to prevent the stylet mouthparts from exiting the BITES.

For experiments involving post-blood-meal (PBM) culturing, the BITES was presented to mosquitoes (n=50) as described. The protocol was modified by covering the BITES with a thin, stretched layer of Parafilm™ during the entire time (20 min) that the BITES was presented to mosquitoes. After the mosquito blood meal acquisition period, the BITES was retrieved and incubated for either 0 min, 20 min, or 3 days in cell culture media containing 1% penicillin-streptomycin for post-blood-meal (PBM) imaging. After the incubation period, the BITES was washed with PBS+ to then be fixed, permeabilized, and stained (Sections 2.7 and 2.8).

### 1.6. Fixation, Staining, and Preparation of BITES Constructs

To acquire images before mosquito blood-feeding, the 4-week culture of cellularized Capgel (BITES) was washed 3× with phosphate-buffered saline (PBS+, with calcium and magnesium, ThermoFisher, Waltham, MA, USA). Fixation was performed by incubating the Capgel with 4% paraformaldehyde (PFA, Sigma-Aldrich, St. Louis, MO, USA) for 30 min at room temperature. Next, the BITES was permeabilized with 0.2% Triton X-100 (Sigma-Aldrich, St. Louis, MO, USA) for 15 min, followed by staining the nuclei and actin proteins using NucBlue Live and ActinGreen 488 (ReadyProbe, Invitrogen; Carlsbad, CA, USA), respectively, solvated in PBS+ for 2 h in darkness. After washing 3× and submerging in PBS+, the BITES was kept at 4° C. until imaged.

### 1.7. Confocal Microscopy and Stereomicroscopy

Confocal fluorescence imaging was performed on the Capgel-BITES by transferring them to a glass-bottom petri dish (Fluorodish™-Sigma-Aldrich, St. Louis, MO, USA) with a plastic spatula. A Zeiss 710 laser scanning confocal microscope at 20× and 40× magnification was used to acquire images with the Zen 2010 software (Zeiss; Jena, Germany). The samples were excited with a 405 nm wavelength for NucBlue and 488 nm for ActinGreen 488 and z-stacks were acquired in both channels. Differential interference contrast (DIC) imaging was also performed to view cells in the context of the Capgel-BITES capillary structure. The images were overlaid, and maximum-intensity z-projections, 3D projections, and orthogonal views were processed using ImageJ. DIC movies/videos were captured using confocal microscopy and processed with the Zen 2010 software. Images of mosquito stylet mouthparts inside BITES were taken with a stereo microscope equipped with a digital camera (MU1803, AmScope, Irvine, CA, USA).

### 1.8. Evaluating Nuclear Orientation in BITES Microvessels

To determine the orientation of HDF (n=12 images) and HUVEC (n=6 images) nuclei in capillaries, threshold application was performed on the NucBlue channel maximum projections of z-stacked images taken at cellularized regions perpendicular to the capillaries.

These images were binarized in ImageJ and then processed using the “region props” function in MATLAB (vR2021b, MathWorks, Natick, MA, USA). Major and minor axes of the nuclei were used to approximate the elliptical blobs. When the major axes of nuclei were parallel with respect to the image vertical axis by +20°, nuclear orientation was considered to be aligned with the capillaries. Nuclei with centroids above the image horizontal midline were considered to have orientation values of −90° to 90°, and nuclei with centroids below the midline were considered to have orientations from 90° to 270°.

### 1.9. Statistical Analysis

The median and mean of the number of mosquitoes attempting to feed on the BITES were calculated in Origin 2021 (OriginLab Corporation, Northampton, MA, USA) by analyzing the data in a box chart. Tukey's outlier detection criterion was applied to determine outliers in the data, using the interquartile range (IQR). Values were labeled as outliers if found to be less than

$$minimum = Q 1 - 1.5 IQR \qquad ( 5 )$$

or if greater than

$$maximum = Q 3 - 1.5 IQR \qquad ( 6 )$$

where Q1 is a value that is less than 75% of the points in the dataset, Q3 is a value that is greater than 75% of the points in the dataset, and the difference in these values is used to find

$$IQR = Q 3 - Q 1. \qquad ( 7 )$$

Using the Chi-squared (χ²) test statistic with a 95% confidence interval and two (2) degrees of freedom, the Marascuillo procedure was used as detailed in the NIST e-Handbook of Statistical Methods to determine the statistical significance of percent mosquito blood-feeding at different BITES presentation times [33].

### Example 2. Capgel Blocks

The outcomes for a host bitten by an arthropod are significantly influenced by the early events occurring at the bite site [9]. Therefore, the BITES platform was developed using capillary alginate gel scaffolds, human cells, and blood (FIG. 1) as a new tool to investigate mosquito biting and blood-feeding.

The 3D microstructures of uniaxial capillaries running in parallel make Capgel an ideal scaffold to template the growth of cultured human cells into hollow tubular microvessel-like structures that can be filled with blood to form a BITES construct (FIG. 1A-D). The hydrogel nature of Capgel supports the gentle heating of the construct to a physiological temperature range, i.e., 34-37° C., and is posited to allow mosquitoes to naturally bite/penetrate, probe for, and blood-feed from the stacked array of blood-filled BITES microvessel structures (FIG. 1E and inset).

Further, the BITES constructs can then be recovered following arthropod biting/probing/blood-feeding and transferred into media to incubate for additional time in cell culture if desired (FIG. 1F). Ultimately, these BITES constructs are to be retrieved, processed, and analyzed, which here included recovery, fixation, staining, and imaging/microscopy (FIG. 1G). Herein, BITES was tested against Ae. aegypti female mosquitoes as a prototypic biting vector arthropod.

2.1. Blood Meal Acquisition by Ae. Aegypti from Blood-Loaded, Non-Cellularized Capgel

To examine whether the Capgel biomaterials are robust 3D tissue scaffolds for BITES constructs, the loading, distribution, and movement of blood within the Capgel blocks was evaluated, as well as the ability of Ae. aegypti females to bite into, probe, and blood-feed from warmed, blood-loaded Capgel (FIG. 2). The addition of defibrinated blood to the open ends of Capgel capillaries (termed capview, FIG. 2A) rapidly loaded blood into these capillaries and allowed for the visualization of the blood, perpendicular to the capillary long axis, termed raftview (FIG. 2B). This blood was confined to and distributed within the capillaries, did not appear to bind to the Capgel walls (specifically the red blood cells—RBCs), and was able to freely move/flow within these microstructures, observations that match the expectations of microvessels.

Further, when fluid was applied to one end of the capillary, RBCs were observed flowing out of the opposite capillary ends.

The taking of a blood meal by a representative female Ae. aegypti mosquito from a warmed (34-37° C.), blood-loaded Cagpel oriented in raftview is summarized in FIG. 2, panels C-I. This process/event encompasses the following: biting and probing (FIG. 2C); the initiation of blood ingestion (FIG. 2D); feeding to repletion, which included prediuretic droplet excretion/expulsion (FIG. 2E-H) and took a total of approximately three (3) minutes to complete. As blood ingestion proceeded and the midgut filled, the width of the abdomen correspondingly increased (FIG. 2C-H, white lines).

The relative amount of this steady increase was quantified using an abdomen width ratio metric (Equation (1)) and plotted against the blood-feeding time (FIG. 2I, black dots and line). The mosquito began excreting prediuretic fluid in the form of a clear droplet from its anus 18 s after the onset of blood ingestion (FIG. 2E, green circle) and forcibly expelled it across a short distance less than a second later (FIG. 2F, green circle). The female excreted/expelled nine such additional prediuretic droplets (FIG. 2G, H, green circles) at roughly regular intervals over the remaining 146 s of blood-feeding (FIG. 2I, red dots and line). Approximating a droplet volume as a sphere (Equation (4)) and adding these to the calculated estimate for the final volume of the blood in the midgut yielded 2.0 μL for the estimated total volume of blood ingested by the mosquito (FIG. 2I, green number).

2.2. Cellularization of Capgel with HDFs or HUVECs

The uniform capillary microarchitectures of Cagpel scaffolds were cellularized with either HDFs or HUVECs and then characterized (FIG. 3). The Capgel scaffold blocks for cellularization had evenly distributed patent capillaries that were ˜50-70 μm in diameter (FIG. 3A) and ran parallel to each other from end to end (FIG. 3B). Both cell types colonized the Capgel scaffolds during the four-week culture period, especially the HDFs (FIG. 3C-F). Cells attached and spread on the Capgel, ultimately lining the capillary walls with tissue (FIG. 3C, E). After a month in the scaffold culture, HDFs lined most capillaries with continuous, lengthy (>300 μm) tissue structures (FIG. 3D). HUVECs also lined multiple Capgel capillaries during this same time in culture with shorter (<200 μm), patchy segments of tissue (FIG. 3F).

The microvessel-like 3D tissue morphologies formed by the cells during culture in the scaffolds are evident in FIG. 4. Consistent with the FIG. 3 data, the raftview maximum z-projection confocal fluorescent micrographs show the extensive HDF tissue formations throughout the Capgel (FIG. 4A) and the focal structures formed by HUVECs (FIG. 4E). Also consistent are the associated reconstructed orthogonal capviews showing that both HDFs and HUVECs lined the capillary walls to form patent, tubular tissues (FIG. 4B,F, respectively). The dense scaffold cellularization by HDFs is evident in the 3D raft- and capview renderings (FIG. 4C,D, respectively), as is the more diffuse HUVEC cellularization of the Capgel (FIG. 4G, H, respectively).

The engineered microvessel tissues adopted orientations parallel to the long axes of scaffold capillaries (FIG. 5). Confocal fluorescence maximum z-projection micrographs of HDFs and HUVECs stained with NucBlue clearly show elliptically shaped nuclei preferentially elongated in the capillary long-axis direction, i.e., vertically in the images (FIG. 5A, D, respectively). Fluorescent micrographs captured at an increased magnification and overlaid with the associated DIC images show that HDF and HUVEC cell bodies were also elongated preferentially in this same direction (FIG. 5B,E, respectively).

Quantification of cell orientations in the scaffold capillaries revealed that 84% of HDFs and 54% of HUVECs were oriented within +20° of the capillary direction (FIG. 5C, F, respectively).

2.3. Blood Meal Acquisition by Ae. aegypti from BITES Tissue

Blood was easily loaded into BITES tissue (FIG. 6A, white asterisk, raftview orientation) and not restricted by the HDF cellularization. Mosquitoes swarmed the BITES warmed (34-37° C.) constructs, frequently biting, probing, and/or blood-feeding (FIG. 6A).

Warmed BITES constructs were presented to 50 Female Ae. aegypti for 4, 15, or 20 min in independent experiments (FIG. 6B). The mean percentages (±standard deviation-SD) and medians of mosquitoes engaging in blood meal acquisition behaviors on the BITES tissue (i.e., biting, probing, and/or blood-feeding) were 19±4.4%, 20%; 24±4.9%, 23%; and 30±8.2%, 30% for the 4, 15, and 20 min presentations, respectively (FIG. 6B, dots for means, horizontal lines for medians). The interquartile ranges (IQR) increased from 2% for the 4 min presentation to 6.5% for the 15 min presentation and to 10% for the 20 min presentation (FIG. 6B, gray boxes). Outliers—defined as those values outside of Tukey's range—were only observed for the 4 min presentation (FIG. 6B stars, 12% and 24%). All other values observed for the different BITES presentation times fell within the whisker ranges of the box plot (FIG. 6B, vertical brackets). Using the Marascuillo procedure [33], no significant differences were found for any of the mean proportions of females engaging BITES during the different presentation times.

Individual Ae. aegypti had a range of engorgement levels after taking blood meals from the BITES tissue (FIG. 6C). In less than a minute, five of the six mosquitoes analyzed (10% of the total mosquitoes in the cage) had visually detectable midgut distention with abdomen width ratios (Equation (1)) greater than 1.5. The average time to blood “meal desistance” was consistent between the females, with a mean±SD of 151±46 s (FIG. 6C, blue sphere, horizontal error bars). The average level of engorgement (i.e., abdomen width ratio) at this average desistance time was more varied, having a mean±SD of 3.6±1.8, and the average calculated estimate for the final blood volume in a mosquito midgut (Equations (2) and (3)) was 2.1±1.2 μL (FIG. 6C, blue sphere, vertical error bars; upper right corner text).

2.4. In Situ Characterization of Ae. Aegypti Stylet Mouthparts in BITES Microvascular Beds and Post-Blood-Meal BITES Culture

To assess the microscopic interactions of Ae. aegypti fascicles of stylet mouthparts penetrated BITES microvessel beds, mosquitoes were first snap-frozen with liquid nitrogen mid-blood-meal acquisition from BITES tissue cellularized with HDFs. Snap-freezing captured the penetration of the mosquito fascicle/stylet mouthparts into a blood-loaded BITES tissue and fixed it in situ (FIG. 7A).

DIC micrographs and corresponding supplementary videos show that blood was loaded and able to freely move/flow within the microvessel structures of HDF-cellularized BITES tissue (FIG. 7B, C). A series of DIC micrographs at different focal planes of the situation, pictured in FIG. 7A, revealed that the Ae. aegypti pushed the stylet mouthparts through/past multiple layers of blood-filled BITES microvessel structures to acquire the blood meal (FIG. 7D-F, capview orientation; FIG. 7G-I, raftview).

It is conspicuous that areas near the fascicle track and at the end of the stylet mouthparts in the raftview images were devoid of RBCs (FIG. 7G-I, green dashed lines). A 3D-rendered depth profile created by leveraging the autofluorescence of the fascicle in the 488 nm channel indicated that the penetration depth of the stylet mouthparts into the BITES tissue was ˜500 μm (Figure S3B). Another series of DIC micrographs zoomed in to the end of the fascicle show this “interaction volume” in greater detail (FIG. 7J-L), including RBCs inside the labrum and the micropuncture site of a BITES microvessel structure by the stylet mouthparts (FIG. 7J,L, respectively).

To test whether the BITES tissues could be cultured following blood meal acquisitions by Ae. aegypti females, HDF-cellularized BITES tissue was first covered with a thin Parafilm™ membrane and then presented to mosquitoes. Maximum z-projection confocal fluorescence micrographs of these BITES microvessel bed tissues showed that the HDFs and microvessel structures were intact immediately (0 min) post-blood-meal (PBM) as well as after short (20 min) and longer (3 days) times in culture PBM (FIG. 7M-O, respectively). Notably, no obvious signs of contamination, such as yellow cloudy media, aggregates of bacteria, or fungal hyphae, were observed in the 3-day PBM BITES tissue culture.

### raftviewraftview Abbreviations

Ae.—Aedes
BITES—Biologic Interfacial Tissue-Engineered System
Capgel—Capillary alginate gel
DEET—N,N-diethyl-meta-toluamide
DENV—Dengue virus
DMEM—Dulbecco's Modified Eagle Medium
DI—Deionized
DIC—Differential interference contrast
FBS—Fetal bovine serum
FTSE—Full-thickness skin equivalents
HDFs—Human dermal fibroblasts
HUVECs—Human umbilical vein endothelial cells
IQR—Interquartile range
LVES—Large vessel endothelial supplement
PBM—Post-blood-meal
PBS—Phosphate-buffered saline
PFA—Paraformaldehyde
RBC—Red blood cell
SEM—Standard error of the mean
SD—Standard deviation
ZIKV—Zika virus

### REFERENCES

1. WHO. Global Vector Control Response 2017-2030; World Health Organization: Geneva, Switzerland, 2017.
2. Wheeler, N.J.; Heimark, Z. W.; Airs, P. M.; Mann, A.; Bartholomay, L. C.; Zamanian, M. Genetic and functional diversification of chemosensory pathway receptors in mosquito-borne filarial nematodes. PLOS Biol. 2020, 18, e3000723. [CrossRef]
3. GBD Disease Injury Incidence and Prevalence Collaborators. Global, regional, and national incidence, prevalence, and years lived with disability for 354 diseases and injuries for 195 countries and territories, 1990-2017: A systematic analysis for the Global Burden of Disease Study 2017. Lancet 2018, 392, 1789-1858. [CrossRef] [PubMed]
4. GBD DALYs and HALE Collaborators. Global, regional, and national disability-adjusted life-years (DALYs) for 315 diseases and injuries and healthy life expectancy (HALE), 1990-2015: A systematic analysis for the Global Burden of Disease Study 2015. Lancet 2016, 388, 1603-1658. [CrossRef] [PubMed]
5. Agarwal, A.; Joshi, G.; Nagar, D. P.; Sharma, A. K.; Sukumaran, D.; Pant, S. C.; Parida, M. M.; Dash, P. K. Mosquito saliva induced cutaneous events augment Chikungunya virus replication and disease progression. Infect. Genet. Evol. 2016, 40, 126-135. [CrossRef] [PubMed]
6. Briant, L.; Desprès, P.; Choumet, V.; Missé, D. Role of skin immune cells on the host susceptibility to mosquito-borne viruses. Virology 2014, 464-465, 26-32. [CrossRef] [PubMed]
7. Novelo, M.; Hall, M. D.; Pak, D.; Young, P. R.; Holmes, E. C.; McGraw, E. A. Intra-host growth kinetics of dengue virus in the mosquito Aedes aegypti. PLOS Pathog. 2019, 15, e1008218. [CrossRef] [PubMed]
8. Guerrero, D.; Cantaert, T.; Misse, D. Aedes Mosquito Salivary Components and Their Effect on the Immune Response to Arboviruses. Front. Cell Infect. Microbiol. 2020, 10, 407. [CrossRef]
9. Ryman, K. D.; Klimstra, W. B. Host responses to alphavirus infection. Immunol. Rev. 2008, 225, 27-45. [CrossRef]
10. Romano, D.; Stefanini, C.; Canale, A.; Benelli, G. Artificial blood feeders for mosquitoes and ticks—Where from, where to? Acta Trop. 2018, 183, 43-56. [CrossRef] [PubMed]
11. Joint FAO/IAEA Programme. Guidelines for Routine Colony Maintenance of Aedes Mosquito Species; Food and Agriculture Organization of the United Nations, Ed.; International Atomic Energy Agency: Vienna, Austria, 2017; Volume 17.
12. Luo, Y. P. A novel multiple membrane blood-feeding system for investigating and maintaining Aedes aegypti and Aedes albopictus mosquitoes. J. Vector Ecol. 2014, 39, 271-277. [CrossRef]
13. Rutledge, L. C.; Moussa, M. A.; Belletti, C. J. An in vitro blood-feeding system for quantitative testing of mosquito repellents. Mosq. News 1976, 36, 283-293.
14. Ooi, C.-P.; Ahmad, R.; Ismail, Z. Effectiveness of several locally available membranes used for artificial feeding of Aedes albopictus Skuse. Trop. Biomed. 2005, 22, 69-71. [PubMed]
15. Sri-in, C.; Weng, S.-C.; Shiao, S.-H.; Tu, W.-C. A simplified method for blood feeding, oral infection, and saliva collection of the dengue vector mosquitoes. PLOS ONE 2020, 15, e0233618. [CrossRef] [PubMed]
16. Hamel, R.; Dejarnac, O.; Wichit, S.; Ekchariyawat, P.; Neyret, A.; Luplertlop, N.; Perera-Lecoin, M.; Surasombatpattana, P.; Talignani, L.; Thomas, F.; et al. Biology of Zika Virus Infection in Human Skin Cells. J. Virol. 2015, 89, 8880-8896. [CrossRef]
17. Moser, L. A.; Boylan, B. T.; Moreira, F. R.; Myers, L. J.; Svenson, E. L.; Fedorova, N. B.; Pickett, B. E.; Bernard, K. A. Growth and adaptation of Zika virus in mammalian and mosquito cells. PLOS Negl. Trop. Dis. 2018, 12, e0006880. [CrossRef]
18. Wichit, S.; Hamel, R.; Zanzoni, A.; Diop, F.; Cribier, A.; Talignani, L.; Diack, A.; Ferraris, P.; Liegeois, F.; Urbach, S.; et al. SAMHD1 Enhances Chikungunya and Zika Virus Replication in Human Skin Fibroblasts. Int. J. Mol. Sci. 2019, 20, 1695. [CrossRef] [PubMed]
19. Avirutnan, P.; Malasit, P.; Seliger, B.; Bhakdi, S.; Husmann, M. Dengue Virus Infection of Human Endothelial Cells Leads to Chemokine Production, Complement Activation, and Apoptosis. J. Immunol. 1998, 161, 6338-6346. [CrossRef] [PubMed]
20. Esterly, A. T.; Lloyd, M. G.; Upadhyaya, P.; Moffat, J. F.; Thangamani, S. A Human Skin Model for Assessing Arboviral Infections. JID Innov. 2022, 2, 100128. [CrossRef]
21. Limon-Flores, A. Y.; Perez-Tapia, M.; Estrada-Garcia, I.; Vaughan, G.; Escobar-Gutierrez, A.; Calderon-Amador, J.; HerreraRodriguez, S. E.; Brizuela-Garcia, A.; Heras-Chavarria, M.; Flores-Langarica, A.; et al. Dengue virus inoculation to human skin explants: An effective approach to assess in situ the early infection and the effects on cutaneous dendritic cells. Int. J. Exp. Pathol. 2005, 86, 323-334. [CrossRef] [PubMed]
22. Janson, K. D.; Carter, B. H.; Jameson, S. B.; de Verges, J. E.; Dalliance, E. S.; Royse, M. K.; Kim, P.; Wesson, D. M.; Veiseh, O. Development of an automated biomaterial platform to study mosquito feeding behavior. Front. Bioeng. Biotechnol. 2023, 11, 1103748. [CrossRef]
23. Schmid, M. A.; Glasner, D. R.; Shah, S.; Michlmayr, D.; Kramer, L. D.; Harris, E. Mosquito Saliva Increases Endothelial Permeability in the Skin, Immune Cell Migration, and Dengue Pathogenesis during Antibody-Dependent Enhancement. PLOS Pathog. 2016, 12, e1005676. [CrossRef] [PubMed]
24. Reuter, C.; Walles, H.; Groeber, F. Preparation of a Three-Dimensional Full Thickness Skin Equivalent. Methods Mol. Biol. 2017, 1612, 191-198. [CrossRef] [PubMed]
25. Willenberg, B. J.; Hamazaki, T.; Meng, F. W.; Terada, N.; Batich, C. Self-assembled copper-capillary alginate gel scaffolds with oligochitosan support embryonic stem cell growth. J. Biomed. Mater. Res. A 2006, 79, 440-450. [CrossRef] [PubMed]
26. Willenberg, B. J.; Zheng, T.; Meng, F. W.; Meneses, J. C.; Rossignol, C.; Batich, C. D.; Terada, N.; Steindler, D. A.; Weiss, M. D. Gelatinized copper-capillary alginate gel functions as an injectable tissue scaffolding system for stem cell transplants. J. Biomater. Sci. Polym. Ed. 2011, 22, 1621-1637. [CrossRef] [PubMed]
27. Rocca, D. G.; Willenberg, B. J.; Qi, Y.; Simmons, C. S.; Rubiano, A.; Ferreira, L. F.; Huo, T.; Petersen, J. W.; Ruchaya, P. J.; Wate, P. S.; et al. An injectable capillary-like microstructured alginate hydrogel improves left ventricular function after myocardial infarction in rats. Int. J. Cardiol. 2016, 220, 149-154. [CrossRef] [PubMed]
28. Bosak, A.; Kwan, M. W. C.; Willenberg, A.; Perle, K. M. D. L.; Weinstein, D.; Hines, R. B.; Schultz, G. S.; Ross, E. A.; Willenberg, B. J. Capillary alginate gel (Capgel™) for the treatment of full-thickness dermal wounds in a hypoxic mouse model. Int. J. Polym. Mater. Polym. Biomater. 2018, 68, 1108-1117. [CrossRef]
29. Anderson, W. A.; Willenberg, A. R.; Bosak, A. J.; Willenberg, B. J.; Lambert, S. Use of a capillary alginate gel (Capgel™) to study the three-dimensional development of sensory nerves reveals the formation of a rudimentary perineurium. J. Neurosci. Methods 2018, 305, 46-53. [CrossRef]
30. George, D. S.; Anderson, W. A.; Sommerhage, F.; Willenberg, A. R.; Hines, R. B.; Bosak, A. J.; Willenberg, B. J.; Lambert, S. Bundling of axons through a capillary alginate gel enhances the detection of axonal action potentials using microelectrode arrays. J. Tissue Eng. Regen. Med. 2019, 13, 385-395. [CrossRef]
31. Panarello, A. P.; Seavey, C. E.; Doshi, M.; Dickerson, A. K.; Kean, T. J.; Willenberg, B. J. Transforming Capillary Alginate Gel (Capgel) into New 3D-Printing Biomaterial Inks. Gels 2022, 8, 376. [CrossRef]
32. Doshi, M.; Bosak, A.; Neal, C. J.; Isis, N.; Kumar, U.; Jeyaranjan, A.; Sakthivel, T. S.; Singh, S.; Willenberg, A.; Hines, R. B.; et al. Exposure to nanoceria impacts larval survival, life history traits and fecundity of Aedes aegypti. PLOS Negl. Trop. Dis. 2020, 14, e0008654. [CrossRef]
33. National Institute of Standards and Technology. Chapter 7 Product and Process. Comparisons; Department of Commerce: Washington, DC, USA, 2012. [CrossRef]
34. Ribeiro, J. M.; Rossignol, P. A.; Spielman, A. Aedes aegypti: Model for Blood Finding Strategy and Prediction of Parasite Manipulation. Exp. Parasitol. 1985, 60, 118-132. [CrossRef] [PubMed]
35. Friuli, M.; Cafarchia, C.; Lia, R. P.; Otranto, D.; Pombi, M.; Demitri, C. From tissue engineering to mosquitoes: Biopolymers as tools for developing a novel biomimetic approach to pest management/vector control. Parasit. Vectors 2022, 15, 79. [CrossRef] [PubMed]
36. Kumar, S.; Hol, F. J. H.; Pujhari, S.; Ellington, C.; Narayanan, H. V.; Li, H.; Rasgon, J. L.; Prakash, M. A microfluidic platform for highly parallel bite by bite profiling of mosquito-borne pathogen transmission. Nat. Commun. 2021, 12, 6018. [CrossRef]
37. Chaves, L. F.; Harrington, L. C.; Keogh, C. L.; Nguyen, A. M.; Kitron, U. D. Blood feeding patterns of mosquitoes: Random or structured? Front. Zool. 2010, 7, 3. [CrossRef] [PubMed]
38. Choo, Y. M.; Buss, G. K.; Tan, K.; Leal, W. S. Multitasking roles of mosquito labrum in oviposition and blood feeding. Front. Physiol. 2015, 6, 306. [CrossRef] [PubMed]
39. Krenn, H. W.; Aspock, H. Form, function and evolution of the mouthparts of blood-feeding Arthropoda. Arthropod Struct. Dev. 2012, 41, 101-118. [CrossRef]
40. Clement, A. N. Chapter 11: Adult Food and Feeding Mechanisms; The Biology of Mosquitoes; Chapman and Hall: London, UK; Cambridge, MA, USA, 1992; Volume 1, pp. 220-250.
41. Arca, B.; Lombardo, F.; Valenzuela, J. G.; Francischetti, I. M.; Marinotti, O.; Coluzzi, M.; Ribeiro, J. M. An updated catalogue of salivary gland transcripts in the adult female mosquito, Anopheles gambiae. J. Exp. Biol. 2005, 208 Pt 20, 3971-3986. [CrossRef]
42. Vogt, M. B.; Lahon, A.; Arya, R. P.; Kneubehl, A. R.; Spencer Clinton, J. L.; Paust, S.; Rico-Hesse, R. Mosquito saliva alone has profound effects on the human immune system. PLOS Negl. Trop. Dis. 2018, 12, e0006439. [CrossRef]
43. Nayar, J. K.; Sauerman, D. M. The Effects of Nutrition on Survival and Fecundity in Florida Mosquitoes: Part 2. Utilization of a blood meal for survival. J. Med. Entomol. 1975, 12, 99-103. [CrossRef]
44. Briegel, H.; Rezzonico, L. Concentration of Host Blood Protein During Feeding by Anopheline Mosquitoes (Diptera: Culicidae). J. Med. Entomol. 1985, 22, 612-618. [CrossRef]
45. Beyenbach, K. W.; Petzel, D. H. Diuresis in Mosquitoes: Role of a Natriuretic Factor. Am. Physiol. Soc. 1987, 2, 171-175. [CrossRef]
46. Lahondère, C.; Lazzari, C. R. Mosquitoes Cool Down during Blood Feeding to Avoid Overheating. Curr. Biol. 2012, 22, 40-45. [CrossRef] [PubMed]
47. Stobbart, R. H. The control of the diuresis following a blood meal in females of the yellow fever mosquito aedes Aegypti (L.). J. Exp. Biol. 1977, 69, 53-85. [CrossRef] [PubMed]
48. Braverman, I. M. The Cutaneous Microcirculation. J. Investig. Dermatol. Symp. Proc. 2000, 5, 3-9. [CrossRef] [PubMed]
49. Kosyakova, N.; Kao, D. D.; Figetakis, M.; Lopez-Giraldez, F.; Spindler, S.; Graham, M.; James, K. J.; Won Shin, J.; Liu, X.; Tietjen, G. T.; et al. Differential functional roles of fibroblasts and pericytes in the formation of tissue-engineered microvascular networks in vitro. NPJ Regen. Med. 2020, 5, 1. [CrossRef] [PubMed]
50. Hetheridge, C.; Mavria, G.; Mellor, H. Uses of the in vitro endothelial-fibroblast organotypic co-culture assay in angiogenesis research. Biochem. Soc. Trans. 2011, 39, 1597-1600. [CrossRef] [PubMed]
51. Berthod, F. Chapter 7. Fibroblasts and Endothelial Cells: The Basic Angiogenic Unit; Angiogenesis: Insights from a Systematic Overview; Nova Science Publishers: Hauppauge, NY, USA, 2013; pp. 145-157.
52. Kunz-Schughart, L. A.; Schroeder, J. A.; Wondrak, M.; van Rey, F.; Lehle, K.; Hofstaedter, F.; Wheatley, D. N. Potential of fibroblasts to regulate the formation of three-dimensional vessel-like structures from endothelial cells in vitro. Am. J. Physiol. Cell Physiol. 2006, 290, C1385-C1398. [CrossRef]
53. Uwamori, H.; Ono, Y.; Yamashita, T.; Arai, K.; Sudo, R. Comparison of organ-specific endothelial cells in terms of microvascular formation and endothelial barrier functions. Microvasc. Res. 2019, 122, 60-70. [CrossRef] [PubMed]
54. Langille, B. L.; Adamson, S. L. Relationship between Bloow Flow Direction and Endothelial Cell Orientation at Arterial Branch Sites in Rabbits and Mice. Circ. Res. 1981, 48, 481-488. [CrossRef] [PubMed]
55. Tkachenko, E.; Gutierrez, E.; Saikin, S. K.; Fogelstrand, P.; Kim, C.; Groisman, A.; Ginsberg, M. H. The nucleus of endothelial cell as a sensor of blood flow direction. Biol. Open 2013, 2, 1007-1012. [CrossRef]
56. Vion, A.-C.; Perovic, T.; Petit, C.; Hollfinger, I.; Bartels-Klein, E.; Frampton, E.; Gordon, E.; Claesson-Welsh, L.; Gerhardt, H. Endothelial Cell Orientation and Polarity Are Controlled by Shear Stress and VEGF Through Distinct Signaling Pathways. Front. Physiol. 2021, 11, 623769. [CrossRef]
57. Sinha, R.; Le Gac, S.; Verdonschot, N.; van den Berg, A.; Koopman, B.; Rouwkema, J. Endothelial cell alignment as a result of anisotropic strain and flow induced shear stress combinations. Sci. Rep. 2016, 6, 29510. [CrossRef]
58. Aubin, H.; Nichol, J. W.; Hutson, C. B.; Bae, H.; Sieminski, A. L.; Cropek, D. M.; Akhyari, P.; Khademhosseini, A. Directed 3D cell alignment and elonga.ation in microengineered hydrogels. Biomaterials 2010, 31, 6941-6951. [CrossRef]
59. Calcutt, J. J.; Roberts, M. S.; Anissimov, Y. G. Predicting Viable Skin Concentration: Modelling the Subpapillary Plexus. Pharm. Res. 2022, 39, 783-793. [CrossRef] [PubMed]
60. Le Coupanec, A.; Babin, D.; Fiette, L.; Jouvion, G.; Ave, P.; Misse, D.; Bouloy, M.; Choumet, V. Aedes Mosquito Saliva Modulates Rift Valley Fever Virus Pathogenicity. PLOS Negl. Trop. Dis. 2013, 7, e2237. [CrossRef] [PubMed]
61. Pingen, M.; Bryden, S. R.; Pondeville, E.; Schnettler, E.; Kohl, A.; Merits, A.; Fazakerley, J. K.; Graham, G. J.; McKimmie, C. S. Host Inflammatory Response to Mosquito Bites Enhances the Severity of Arbovirus Infection. Immunity 2016, 44, 1455-1469. [CrossRef] [PubMed]
62. Styer, L. M.; Lim, P. Y.; Louie, K. L.; Albright, R. G.; Kramer, L. D.; Bernard, K. A. Mosquito saliva causes enhancement of West Nile virus infection in mice. J. Virol. 2011, 85, 1517-1527. [CrossRef] [PubMed]
63. Surasombatpattana, P.; Ekchariyawat, P.; Hamel, R.; Patramool, S.; Thongrungkiat, S.; Denizot, M.; Delaunay, P.; Thomas, F.; Luplertlop, N.; Yssel, H.; et al. Aedes aegypti saliva contains a prominent 34-kDa protein that strongly enhances dengue virus replication in human keratinocytes. J. Investig. Dermatol. 2014, 134, 281-284. [CrossRef]
64. Onlamoon, N.; Noisakran, S.; Hsiao, H. M.; Duncan, A.; Villinger, F.; Ansari, A. A.; Perng, G. C. Dengue virus-induced hemorrhage in a nonhuman primate model. Blood 2010, 115, 1823-1834. [CrossRef]
65. Mores, C. N.; Christofferson, R. C.; Davidson, S. A. The role of the mosquito in a dengue human infection model. J. Infect. Dis. 2014, 209 (Suppl. 2), S71-S78. [CrossRef]
66. Leitner, W. W.; Costero-Saint Denis, A.; Wali, T. Immunological consequences of arthropod vector-derived salivary factors. Eur. J. Immunol. 2011, 41, 3396-3400. [CrossRef] [PubMed]
67. Cevc, G.; Vierl, U. Spatial distribution of cutaneous microvasculature and local drug clearance after drug application on the skin. J. Control Release 2007, 118, 18-26. [CrossRef] [PubMed]
68. Sorrell, J. M.; Caplan, A. I. Fibroblast heterogeneity: More than skin deep. J. Cell Sci. 2004, 117 Pt 5, 667-675. [CrossRef] [PubMed]
69. Silva, T. L. A.; Radtke, A.; Balaban, A.; Pascini, T. V.; Pala, Z. R.; Roth, A.; Alvarenga, P. H.; Jeong, Y. J.; Olivas, J.; Ghosh, A. K.; et al. The fibrinolytic system enables the onset of Plasmodiuminfection in the mosquito vector and the mammalian host. Sci. Adv. 2021, 7, eabe3362. [CrossRef] [PubMed]
70. Dormont, L.; Mulatier, M.; Carrasco, D.; Cohuet, A. Mosquito Attractants. J. Chem. Ecol. 2021, 47, 351-393. [CrossRef] [PubMed]
71. Seavey, C. E.; Doshi, M.; Colamarino, A.; Kim, B. N.; Dickerson, A. K.; Willenberg, B. J. Graded Atmospheres of Volatile Pyrethroid Overlaid on Host Cues can be Established and Quantified within a Novel Flight Chamber for Mosquito Behavior Studies. Environ. Entomol. 2023, 52, 197-209. [CrossRef] [PubMed]

What is claimed is:

1. An arthropod bite model comprising a gel platform comprising a plurality of parallel microtubular capillaries,
wherein the gel comprises at least one biopolymer selected from a group comprising alginate, gelatin, collagen, hyaluronic acid, chitosan, laminin, fibronectin, glycosaminoglycans, chondroitin sulfate, dermatan sulfate, proteoglycans or other extracellular matrix molecules, silicones, methacrylate hydrogels, degradable polyester biomaterials (e.g., PLA, PGA, PLLA, etc.), the like and combination thereof, and in particular alginate and gelatin, and
wherein the capillaries are cellularized with a plurality of cells.

2. The arthropod bite model of claim 1, wherein the diameters and lengths of each of the microtubular capillaries may be the same, similar or vary.

3. The arthropod bite model of claim 1, wherein the diameter of a microtubular capillary may be the same, similar or vary between one end and the other end of the capillary.

4. The arthropod bite model of claim 1, wherein the cross-section of each of the microtubular capillaries is circular or non-circular.

5. The arthropod bite model of claim 1, wherein some of the microtubular capillaries are open at both ends and the others are open at one end.

6. The arthropod bite model of claim 1, wherein the diameter of each of the microtubular capillaries is between about 10 μm to about 300 μm, in particular between about 50 and 70 μm.

7. The arthropod bite model of any of claims 1-6, wherein the cell type is at least one selected from dermal fibroblasts, vascular endothelial cells, dermal keratinocytes, immune cells, and mesenchymal stem cells, wherein the immune cells can be macrophages, either tissue-resident or derived from infiltration of monocytes, and/or dendritic cells.

8. The arthropod bite model of any of claims 1-7, wherein the microtubular capillaries are cellularized with dermal fibroblasts, optionally human dermal fibroblasts, alone or with other cell types, optionally vascular endothelial cells, keratinocytes, and/or macrophages.

9. The arthropod bite model of any of claims 1-8, wherein the microtubular capillaries are cellularized with vascular endothelial cells, optionally human umbilical vein endothelial cells, alone or with other cell types, optionally dermal fibroblasts, keratinocytes, and/or macrophages.

10. The arthropod bite model of any one of claims 1-9, wherein the microtubular capillaries are loaded with cell culture medium, serum and/or blood, and wherein optionally the capillaries having open ends at both sides can be connected to a peristaltic pump or height-adjusted syringes to make the cell culture medium, serum and/or blood flow through the microtubular capillaries.

11. A method of making the arthropod bite model of claim 1, comprising steps of:
a. dissolving biopolymer, optionally gelatin, in distilled and deionized water;
b. degrading the biopolymer solution and equilibrate the solution to room temperature;
c. adding alginate to the biopolymer solution to form an alginate/biopolymer solution;
d. disposing the alginate/biopolymer solution in a container coated with dehydrated alginate;
e. covering the container with a porous cover layer soaked with copper (II) sulfate solution;
f. applying copper sulfate solution dropwise to the alginate/biopolymer solution, optionally through the porous cover layer;
g. allowing the alginate/biopolymer solution to set into a capillary alginate gel (Capgel) comprising capillaries; and
h. cutting the Capgel into segments; and
i. optionally, crosslinking the Capgel segments via carbodiimide chemistry.

12. The method of claim 11, further comprising cellularizing the Capgel, wherein Capgel is cellularized by steps of:
a. dissociating about 80% confluent cells in a cell culture container with trypsin;
b. adding serum-containing cell culture medium to the cell culture container having trypsinized cells;
c. moving the contents of the cell culture container into a tube for centrifugation;
d. discarding supernatant after centrifugation to get a cell pellet;
e. adding a small volume of the cell culture medium to the cell pellet and suspending the pellet;
f. calculating cell number;
g. applying the cells to the capillaries of the Capgel;
h. placing the Capgel with capillaries in the vertical direction in a microcentrifuge tube and centrifuging the tube;
i. placing the cell-seeded Capgels into the incubator to allow cell attachment;
j. repeating steps of g, h, and i two times or more times so that enough number of cells are seeded per Capgel block;
k. adding the cell culture medium by submerging Capgel blocks into the cell culture medium; and
l. culturing the cells within the Capgel blocks;
wherein after cell culture, optionally the cell culture medium can be replaced with host animal's serum or blood for mosquito behavior observation.

13. The method of claim 12, wherein the cell type is at least one selected from dermal fibroblasts, vascular endothelial cells, keratinocytes, immune cells, and mesenchymal stem cells, wherein immune cells optionally comprise macrophages, either tissue-resident or derived from infiltration of monocytes, or dendritic cells.

14. The method of claim 13, wherein the microtubular capillaries are cellularized with dermal fibroblasts, optionally human dermal fibroblasts, alone or with other cell types, optionally vascular endothelial cells, keratinocytes, and/or macrophages.

15. The method of claim 13, wherein the microtubular capillaries are cellularized with vascular endothelial cells, optionally human umbilical vein endothelial cells, alone or with other cell types, optionally dermal fibroblasts, keratinocytes, and/or macrophages.

16. The method of any one of claims 11-15, wherein the microtubular capillaries are loaded with cell culture medium, serum and/or blood, and wherein optionally the capillaries having open ends at both sides can be connected to a peristaltic pump or height-adjusted syringes to make the cell culture medium, serum and/or blood flow through the microtubular capillaries.

17. A method of using the arthropod bite model of any one of claims 1-10, the method comprising coating the gel with an insecticide or a repellant to be tested, and subjecting the gel to one or more arthropods.

18. The method of claim 17, wherein the method further comprising observing and/or video-recording behavior of the one or more arthropods.

19. A method for making an arthropod bite model gel platform comprising a plurality of parallel microtubular capillaries, the method comprising steps of:
a. 3-D printing a biopolymer gel, wherein the biopolymer gel comprises a biomaterial, and, optionally, cells,
b. curing the biopolymer gel,
c. if cells are not included in step a, adding cells to the gel capillaries later according to claim 12; and
d. delivering cell culture medium, serum and/or blood to the cellularized capillaries according to claim 12.

20. The method of claim 20, wherein the gel comprises at least one biopolymer selected from a group comprising alginate, gelatin, collagen, hyaluronic acid, chitosan, laminin, fibronectin, glycosaminoglycans, chondroitin sulfate, dermatan sulfate, proteoglycans or other extracellular matrix molecules, silicones, methacrylate hydrogels, degradable polyester biomaterials (e.g., PLA, PGA, PLLA, etc.) and the like, and optionally alginate and gelatin.

21. The method of claim 19, wherein the cell type is at least one selected from dermal fibroblasts, vascular endothelial cells, keratinocytes, immune cells, and mesenchymal stem cells, wherein immune cells optionally comprise macrophages, either tissue-resident or derived from infiltration of monocytes, or dendritic cells, and in particular dermal fibroblasts and/or vascular endothelial cells, and in more particular human dermal fibroblasts and/or human umbilical vein endothelial cells.

22. The arthropod bite model of any of claims 1-10, wherein the microtubular capillaries are loaded with a sugar, sugar alcohol, an alcohol and/or an insecticide, or a solution containing one or more thereof.

23. An arthropod bite model comprising a gel platform comprising a plurality of parallel microtubular capillaries,
wherein the gel comprises at least one biopolymer selected from a group comprising alginate, gelatin, collagen, hyaluronic acid, chitosan, laminin, fibronectin, glycosaminoglycans, chondroitin sulfate, dermatan sulfate, proteoglycans or other extracellular matrix molecules, silicones, methacrylate hydrogels, degradable polyester biomaterials (e.g., PLA, PGA, PLLA, etc.), the like and combination thereof, and in particular alginate and gelatin, and wherein the microtubular capillaries are loaded with a sugar, sugar alcohol, an alcohol and/or an insecticide, or a solution containing one or more thereof.

24.
